SEQUENCE LISTING (31 sequences)

SEQ ID NO: 1 - DNA, 1305 bp, Bacillus species
  CDS (1)..(1302): GH8 xylanase
  sig_peptide (1)..(81)
  mat_peptide (82)..(1302)
atgaagaaaccgttggggaaaattgtcgcaagcaccgcactactcatttctgttgcttttagttcatcgatcgcatcggctgctgtacattcaaagactccagacatccttggcacaactggcaagaacaaccttaaccaggcttacaagaagtatttcgacacaaagggcgacggcaaaggtggctctctttttcactatatgaaggacggctctgcttacatcgcttctacaacagacgacaaccttcttggcaacggctattactctgtaaagactgagggcatgtcatacggcatgatgatcacgcttcaaatgaacgacgagtacaagtttcaaaagctttgggacttcgttcgcaagtacatgcgccattcagaccgcaacgacagcctttacggctatcactcttggcatatgaaaacaaacggctcagacgttcaaacgatcgaccaaaacgttgcgtcagacggcgaggtatggtttgcagctgcacttatgatggcttctggtcgctggggtgacaagaagtacccttacgactacaaggcacgcgctcaagacatgcttgacgcacttgcaggcgacggcgagtacgctaacacaggcaaggagtctcgcgttttcatcaaaaacagcaaggaccaacgctatgctatggtacgctttggcccttacgttaactggactgacccatcttatcatgttccggctttcttcgagcttttcgccaagtcagctaagtcatcacaacaatacttctggaaggacgcagctaacaaatctcgcacgtaccttagcgagacaactttcaagtctgtacttaacaacggctcaacagttactaacgctgctactggcctttttcctgacgaggctggctttgacggcgtttcagacgctgctcattcttcaacagagacagaccgcaacttctcttacgacgcttggcgcactgtatctcatatcgctatggaccatactctttggagctcagctgacaacgcatatcgcgcatctgagcaaaaggctgttaacaagtttcttacattcatgaagcgcgagaactatggtcgcactgcccatgagtatacacttaacggcacagcagtaaagaaaggctctcctgttggccttatcgcagcaaacgctggtggcgcaacggcagcatctgacgcatcacttcgcacgggcttcgctaacgcgtttaactctacatacatcccagaggactactacggctcttgtctttacatgcttaactctcttgttgcgaacggcaagttcgcaatgtatcttccataa

SEQ ID NO: 2 - protein, 434 aa, Bacillus species
MKKPLGKIVASTALLISVAFSSSIASAAVHSKTPDILGTTGKNNLNQAYKKYFDTKGDGKGGSLFHYMKDGSAYIASTTDDNLLGNGYYSVKTEGMSYGMMITLQMNDEYKFQKLWDFVRKYMRHSDRNDSLYGYHSWHMKTNGSDVQTIDQNVASDGEVWFAAALMMASGRWGDKKYPYDYKARAQDMLDALAGDGEYANTGKESRVFIKNSKDQRYAMVRFGPYVNWTDPSYHVPAFFELFAKSAKSSQQYFWKDAANKSRTYLSETTFKSVLNNGSTVTNAATGLFPDEAGFDGVSDAAHSSTETDRNFSYDAWRTVSHIAMDHTLWSSADNAYRASEQKAVNKFLTFMKRENYGRTAHEYTLNGTAVKKGSPVGLIAANAGGATAASDASLRTGFANAFNSTYIPEDYYGSCLYMLNSLVANGKFAMYLP

SEQ ID NO: 3 - protein, 407 aa, Bacillus species
AVHSKTPDILGTTGKNNLNQAYKKYFDTKGDGKGGSLFHYMKDGSAYIASTTDDNLLGNGYYSVKTEGMSYGMMITLQMNDEYKFQKLWDFVRKYMRHSDRNDSLYGYHSWHMKTNGSDVQTIDQNVASDGEVWFAAALMMASGRWGDKKYPYDYKARAQDMLDALAGDGEYANTGKESRVFIKNSKDQRYAMVRFGPYVNWTDPSYHVPAFFELFAKSAKSSQQYFWKDAANKSRTYLSETTFKSVLNNGSTVTNAATGLFPDEAGFDGVSDAAHSSTETDRNFSYDAWRTVSHIAMDHTLWSSADNAYRASEQKAVNKFLTFMKRENYGRTAHEYTLNGTAVKKGSPVGLIAANAGGATAASDASLRTGFANAFNSTYIPEDYYGSCLYMLNSLVANGKFAMYLP

SEQ ID NO: 4 - DNA, 1290 bp, Dyella species
  CDS (1)..(1287): GH8 xylanase
  sig_peptide (1)..(63)
  mat_peptide (64)..(1287)
atgcgcaaatccgcccctatcttctgtctcctgcttctggccacgtcgttgggttcgctggctcaggacagtccggagcaggatgctagggcggtgggtgccgtcgctaccggccattacccggatctgtttgccgaagcaggccacacccgctctgacgtggacaaaaagattcaggcagcctttcaacaactgttccacggcgaccctgccacgcaggcggtctatcacgaggcgggcagtaacgcggacgggccactggcttacatccacgacgtcaacaatgacgatgtgcgctcggaaggcatctcctacgggatgatgattgccgtacaactggaccgcaaacgagagtttgatgcgctctggaactgggcgatgactcacatgtaccagcgcgacccgtcgcatccgtcgtacggctatttcgcctggtcggtgcgtaccgacggcatggtcaatgatccgatgcctgcgcccgatggggaggagtacctgatcaccgcgctgtacttcgccgccgcgcgctggggtaacggtcatgacctctatgactaccgcgcccaggctgatcgcctgctccgcaacgtgctgcaccgggaggccatcaccggccaaaccatccacggtcaaatgacggcactcagcctgttcgatcccaagaccaagatggtccgctttacgcctgaccttgcgaacgccgcgcacaccgatgcgtcctatcatttgccggctttctacgaggtctgggcccgggttgggccgcagtccgaccgcgtgttctggcaccaggctgcgcaggtgagccgcgactatctggcaaccacggcgcacccaaagaccgcgcttactcccgattacggcaacgtcgacggcaccccatgggcggcaccatggcgtccggattctgcggacttccgttacgacgcctggcgcagcgcgatgaattggtcggtggatggatcatggtgggcacaggaccaccgcgaagtcgagatgagcaaccgcctgcaggcattcttcgatgcgcaaggcatggatgcctatcagagcctgtacaagctggacggtaccctcctgggcggcggccacgtcaccggcctgatcgccaccaacgcggtcgccagcctgaccgctaccgatccgcgcaggttggccttcgtccagacattgtgggaacagccggtgcccagtggaccgcagcgctactacaacggcatgctttatttgatggccttgctccattgcagcggcgatttccgcgcatggctgccgcatccatcggcctga

SEQ ID NO: 5 - protein, 429 aa, Dyella species
MRKSAPIFCLLLLATSLGSLAQDSPEQDARAVGAVATGHYPDLFAEAGHTRSDVDKKIQAAFQQLFHGDPATQAVYHEAGSNADGPLAYIHDVNNDDVRSEGISYGMMIAVQLDRKREFDALWNWAMTHMYQRDPSHPSYGYFAWSVRTDGMVNDPMPAPDGEEYLITALYFAAARWGNGHDLYDYRAQADRLLRNVLHREAITGQTIHGQMTALSLFDPKTKMVRFTPDLANAAHTDASYHLPAFYEVWARVGPQSDRVFWHQAAQVSRDYLATTAHPKTALTPDYGNVDGTPWAAPWRPDSADFRYDAWRSAMNWSVDGSWWAQDHREVEMSNRLQAFFDAQGMDAYQSLYKLDGTLLGGGHVTGLIATNAVASLTATDPRRLAFVQTLWEQPVPSGPQRYYNGMLYLMALLHCSGDFRAWLPHPSA

SEQ ID NO: 6 - protein, 408 aa, Dyella species
QDSPEQDARAVGAVATGHYPDLFAEAGHTRSDVDKKIQAAFQQLFHGDPATQAVYHEAGSNADGPLAYIHDVNNDDVRSEGISYGMMIAVQLDRKREFDALWNWAMTHMYQRDPSHPSYGYFAWSVRTDGMVNDPMPAPDGEEYLITALYFAAARWGNGHDLYDYRAQADRLLRNVLHREAITGQTIHGQMTALSLFDPKTKMVRFTPDLANAAHTDASYHLPAFYEVWARVGPQSDRVFWHQAAQVSRDYLATTAHPKTALTPDYGNVDGTPWAAPWRPDSADFRYDAWRSAMNWSVDGSWWAQDHREVEMSNRLQAFFDAQGMDAYQSLYKLDGTLLGGGHVTGLIATNAVASLTATDPRRLAFVQTLWEQPVPSGPQRYYNGMLYLMALLHCSGDFRAWLPHPSA

SEQ ID NO: 7 - DNA, 1305 bp, Bacillus licheniformis
  CDS (1)..(1302): GH8 xylanase
  sig_peptide (1)..(84)
  mat_peptide (85)..(1302)
atgcgaaaaagtttaaaatggatcatggctgtctttattggtttaacatgtttttgtgccgcttattctcaaacagccatggcagccgtacattccaagactccggatattctcggaacaacgggtaaaaacaatttgaatcaggcctataaaaaatactttgatacaaaaggcgatggtaaaggcggatcgctgtttcattatatgaaagacggttcagcttacatcgcttcgacgacggacgataacctgggcaatggctactacagcgtcaaaacagagggtatgagttacgggatgatgattacgctgcaaatgaatgatgagtataagttccaaaagctgtgggatttcgtccgtaaatatatgcgccattccgacagaaacgacagtttgtacggctaccatagctggcatatgaaaacgaacggctcagacgtgcaaaccattgatcagaatgttgcatctgacggcgaggtctggtttgcggcagcgctcatgatggcatctggccgctggggagataaaaaatacccgtacgattacaaagagcgggctcaagacatgcttgacgctttggccggtgatggagaatacgccaacaccggtaaggaatcgcgagtgtttattaaaaacagcaaggatcagcgctatgcgatggttcgattcggcccttatgtcaattggacggacccatcctaccacgttcctgcttttttcgagttgttcgctaaaagcgccagaagcagccagcagtatttttggaaagatgcagcaaacaaatctcgaacatacttgtcagaaacaacgtttaaaagcgtgttaaataacggatcaaccgtcaccaatgcggccacagggctgtttcccgacgaagccggctttgacggcgtaagcgatgcggctcattcctcaacggaaacagaccgaaatttcagctatgatgcttggcggacggtgtctcatatcgctatggatcatacactttggtcctcggcagataacgcatatcgcgccagcgagcaaaaagcggttaacaagttcttgacgtttatgaaacgggaaaactacgggagaacggcccatgaatatacgctgaatgggacggccgtcaaaaaaggaagcccggttggcctgatcgccgccaatgcgggcggcgcgactgcagcaagcgatgcatcattaaggacgggatttgccaatgcgtttaacagcacatatattccagaagattattacggatcatgtctttacatgctgaacagccttgtggcaaacggcaaatttgcgatgtaccttccttaa

SEQ ID NO: 8 - protein, 434 aa, Bacillus licheniformis
MRKSLKWIMAVFIGLTCFCAAYSQTAMAAVHSKTPDILGTTGKNNLNQAYKKYFDTKGDGKGGSLFHYMKDGSAYIASTTDDNLGNGYYSVKTEGMSYGMMITLQMNDEYKFQKLWDFVRKYMRHSDRNDSLYGYHSWHMKTNGSDVQTIDQNVASDGEVWFAAALMMASGRWGDKKYPYDYKERAQDMLDALAGDGEYANTGKESRVFIKNSKDQRYAMVRFGPYVNWTDPSYHVPAFFELFAKSARSSQQYFWKDAANKSRTYLSETTFKSVLNNGSTVTNAATGLFPDEAGFDGVSDAAHSSTETDRNFSYDAWRTVSHIAMDHTLWSSADNAYRASEQKAVNKFLTFMKRENYGRTAHEYTLNGTAVKKGSPVGLIAANAGGATAASDASLRTGFANAFNSTYIPEDYYGSCLYMLNSLVANGKFAMYLP

SEQ ID NO: 9 - protein, 406 aa, Bacillus licheniformis
AVHSKTPDILGTTGKNNLNQAYKKYFDTKGDGKGGSLFHYMKDGSAYIASTTDDNLGNGYYSVKTEGMSYGMMITLQMNDEYKFQKLWDFVRKYMRHSDRNDSLYGYHSWHMKTNGSDVQTIDQNVASDGEVWFAAALMMASGRWGDKKYPYDYKERAQDMLDALAGDGEYANTGKESRVFIKNSKDQRYAMVRFGPYVNWTDPSYHVPAFFELFAKSARSSQQYFWKDAANKSRTYLSETTFKSVLNNGSTVTNAATGLFPDEAGFDGVSDAAHSSTETDRNFSYDAWRTVSHIAMDHTLWSSADNAYRASEQKAVNKFLTFMKRENYGRTAHEYTLNGTAVKKGSPVGLIAANAGGATAASDASLRTGFANAFNSTYIPEDYYGSCLYMLNSLVANGKFAMYLP

SEQ ID NO: 10 - DNA, 2769 bp, Marinimicrobium species
  CDS (1)..(2766): GH8 xylanase incl. N-terminal CBM
  sig_peptide (1)..(66)
  mat_peptide (67)..(2766)
atgaagacaataaaaattcttttcataatcttatgtgccaacttttttgtgcacagcgctcatgccgaactattgcagcagtttgacctggaaaatggccagctgttggcaggctacagcggcgtaatcgactctccgtttttaggggcagcggcttacgcgaatgacgacggcgtagaaacagaagcacatttttccaatatccccggcgttttccgggtggatattcgcggtgcgagcagcaacgacaacactgcgggaatcagcgtgtatctgggggaccggagaatgggtggtgtgagctttgatggctcttcaccctcggtcgaaagcctcgaattcacattggagtctgcaccggattcttcgacgatccgttttattctggaaacggacgacggctccagcaatacttacctggactggtttgaattgcaccgtgtcggtgatattccgccgccccctccaccccccgtactgccttctgaaggcgcctatgagtcgggcgtatatcgcaacctgtttgtggaaatgggtctctccgaggccgaggttgaagctcgggtagaggaggtctatgaacaactgtttcacagccccgatcaggaagacgaagccatttttataccggttggtgatgacatggcgtacatctgggacgtgggcaacaacgacgttcgcagtgaaggcatgtcttacggcatgatgatggcggtccagatggatcgaaaagaggatttcgatcgcctctggaagtgggctcacacctactcgctcaacacctccggggacatgaccggctattttgcctgggaagtgactaccgacggtcaggtccgcgatgccaatccggctcccgatggtgaagcctattttgtgactgcattgttctttgcatcgcacctgtggggtgatggcgagggcatcttcaactacagcgagcaggccaacagcattctggacgatatgtacagcaatggtcagacccgctacaacaaccagggtgagcttgaaaccttcagcctctttgaccacgagacccggctgattgttttctcgccggcaacgcccacggaccgcaactggactgatccttcctatcacctgcctgcttactttgatctgtgggccaagtgggcagacaacaacaaccagttctgggccgaagccgccgaggcaagtcgggagtttctaaaaaccactgtacatccggaaaccggcctgaacccggattatgcccactttgatggcagtccccacggcgatttccagcactggaaagatacctttcaatacgacgcctggcgcaccgtcagcaatgccgccattgactatgcctggttccagaaagatccctggcagatcacctacgcggaaaccctgcaggaattctttgacaacgaaggtattggtgcctattccagcctctatgagctcgacggcaccccctacgagggcaactccgatcattcgcccggtctggtcgccatgaacgcggtggcaagcctggcctcggaagacccccgcgcctggaagtttgtgcaggccctgtgggacacaccggtgcccaccggtcagtggcgctactttgatggcgcactgtatatgttcgccatgctgcgtgccagcggcaactaccgcatcatctgcccggaaggtgagtgcgacttccaggaaccacccagctgcgaagacaacaacacctgcccgagcgaccagcctccggttgctgaagacaacgaggcggtggtcaccgcaggggaatcggtcacagtggatgtcctgagcaatgaccacgaccccgccggcaacaacctttccatcgtcagctatacccagggcggcaacggctctgtgagtgccagcggttcacagctggtctacaccccggacgccgactttgtcgggcaggacagtttcgactacaccgtctcgaatggcagtgctgtcgataacgccactgtttccgtgaccgtggaagcggccgatgatgaagacgacgatgacaatggcaacggaaacggcaacggcgatgagccgggcgctggcgacgatgatgacgaccaggacgtgggttccggctccaacgaattcactcttcgcgccatcggcggctccggcgccgagcaggttcggctgaacctgggcggtgagactctggtcacctggaccctgtccactgactgggaggagtacaccttcagcaccgacgcttcgggtgacctcaatgtcgagctgatcaatgatcaggggcagggctatgaggcccagctggattacctggaggtcaatggtgatcggcgctacgccgaggatcagagctataacaccaccgcctgggatggctattgtggaggcggttcccactccagctggatgcactgcaatggcatgatcgggttcggcgatgtggaccaggccgaggccaatgagtttgtgatctatgccgttggcacctccggtgccgagcagctgcgtctgaacgtggggggtgacaccatcgccaccttcgatctgagcagcagctggcaggagtattccgtgagcgacagcgccaccggtgatatgaacgttgagctgatcaacgatcagggcgagggctatgaggctcgcgttgagtctctcaccgttaacggtgagacccgctacggtgccgaccagagctacaacaccagcgcctggagcggcggctgtgggggcggtagctacaccgactggatgcactgcaacggcatgatcggttttggagacgtcccaaccgccgactga

SEQ ID NO: 11 - protein, 922 aa, Marinimicrobium species
MKTIKILFIILCANFFVHSAHAELLQQFDLENGQLLAGYSGVIDSPFLGAAAYANDDGVETEAHFSNIPGVFRVDIRGASSNDNTAGISVYLGDRRMGGVSFDGSSPSVESLEFTLESAPDSSTIRFILETDDGSSNTYLDWFELHRVGDIPPPPPPPVLPSEGAYESGVYRNLFVEMGLSEAEVEARVEEVYEQLFHSPDQEDEAIFIPVGDDMAYIWDVGNNDVRSEGMSYGMMMAVQMDRKEDFDRLWKWAHTYSLNTSGDMTGYFAWEVTTDGQVRDANPAPDGEAYFVTALFFASHLWGDGEGIFNYSEQANSILDDMYSNGQTRYNNQGELETFSLFDHETRLIVFSPATPTDRNWTDPSYHLPAYFDLWAKWADNNNQFWAEAAEASREFLKTTVHPETGLNPDYAHFDGSPHGDFQHWKDTFQYDAWRTVSNAAIDYAWFQKDPWQITYAETLQEFFDNEGIGAYSSLYELDGTPYEGNSDHSPGLVAMNAVASLASEDPRAWKFVQALWDTPVPTGQWRYFDGALYMFAMLRASGNYRIICPEGECDFQEPPSCEDNNTCPSDQPPVAEDNEAVVTAGESVTVDVLSNDHDPAGNNLSIVSYTQGGNGSVSASGSQLVYTPDADFVGQDSFDYTVSNGSAVDNATVSVTVEAADDEDDDDNGNGNGNGDEPGAGDDDDDQDVGSGSNEFTLRAIGGSGAEQVRLNLGGETLVTWTLSTDWEEYTFSTDASGDLNVELINDQGQGYEAQLDYLEVNGDRRYAEDQSYNTTAWDGYCGGGSHSSWMHCNGMIGFGDVDQAEANEFVIYAVGTSGAEQLRLNVGGDTIATFDLSSSWQEYSVSDSATGDMNVELINDQGEGYEARVESLTVNGETRYGADQSYNTSAWSGGCGGGSYTDWMHCNGMIGFGDVPTAD

SEQ ID NO: 12 - protein, 900 aa, Marinimicrobium species
ELLQQFDLENGQLLAGYSGVIDSPFLGAAAYANDDGVETEAHFSNIPGVFRVDIRGASSNDNTAGISVYLGDRRMGGVSFDGSSPSVESLEFTLESAPDSSTIRFILETDDGSSNTYLDWFELHRVGDIPPPPPPPVLPSEGAYESGVYRNLFVEMGLSEAEVEARVEEVYEQLFHSPDQEDEAIFIPVGDDMAYIWDVGNNDVRSEGMSYGMMMAVQMDRKEDFDRLWKWAHTYSLNTSGDMTGYFAWEVTTDGQVRDANPAPDGEAYFVTALFFASHLWGDGEGIFNYSEQANSILDDMYSNGQTRYNNQGELETFSLFDHETRLIVFSPATPTDRNWTDPSYHLPAYFDLWAKWADNNNQFWAEAAEASREFLKTTVHPETGLNPDYAHFDGSPHGDFQHWKDTFQYDAWRTVSNAAIDYAWFQKDPWQITYAETLQEFFDNEGIGAYSSLYELDGTPYEGNSDHSPGLVAMNAVASLASEDPRAWKFVQALWDTPVPTGQWRYFDGALYMFAMLRASGNYRIICPEGECDFQEPPSCEDNNTCPSDQPPVAEDNEAVVTAGESVTVDVLSNDHDPAGNNLSIVSYTQGGNGSVSASGSQLVYTPDADFVGQDSFDYTVSNGSAVDNATVSVTVEAADDEDDDDNGNGNGNGDEPGAGDDDDDQDVGSGSNEFTLRAIGGSGAEQVRLNLGGETLVTWTLSTDWEEYTFSTDASGDLNVELINDQGQGYEAQLDYLEVNGDRRYAEDQSYNTTAWDGYCGGGSHSSWMHCNGMIGFGDVDQAEANEFVIYAVGTSGAEQLRLNVGGDTIATFDLSSSWQEYSVSDSATGDMNVELINDQGEGYEARVESLTVNGETRYGADQSYNTSAWSGGCGGGSYTDWMHCNGMIGFGDVPTAD

SEQ ID NO: 13 - DNA, 1653 bp, Marinimicrobium species
  CDS (1)..(1650): GH8 xylanase
  sig_peptide (1)..(66)
  mat_peptide (67)..(1650)
atgagacacattcaaaaactattaacggtaagcttgcttagcgctgtgacacattcggctcacgccgaaatactgcaacacacagaactcgaaaatacggctctactgccgggttacaccaacattattgaccagccttttcttggcattgcgacctacgccaacggagatggggcgagtgcggaggccattgtaaataccgcacctggcacctttagattggatgttcgcggcgccagcagcgcttccaatagcgctggcattagcgcttatttagatactcgccgtctcggccagttaagtttttcagggcaaacaccctccgtacaatcactcgagtttgaactggacaacaatagctccgtcgaacaaatacgtttggtgctggaatccgataccggtgccaacgacacactgctcgactggtttgagctgcatcgtataggcgatattccagagccaccgccagcaccggtactgccttctactgggtcttacgaatctggtgtataccgtaatctttttgcggaactgggctacagcaacgcagaggtcgacactaaagttcaatcggcttacgatcaattatttcacagccccgaccaacaaaacgaagcgctgtttgttcccgtaggttcagatatggcttacatctgggatgtcggtaacaacgatgttcgcagtgaaggaatgtcttacggcatgatgatggccgtacaaatgggaagaaaagacgactttgatcgtttatggaagtgggctcacacttattcgctgaataaatcgggcgatatgaaaggttattttgcttggcaagtcagcaccagtggcaatgtactcgataaaaatccagcacccgatggcgaagaatattttgttaccgccttattttttgcctctcacctttggggcgatggcaatggcatttacgattacagcgctcaagctaacagtattcttgatgacctttacagtaacggtcaaacacgctacaacaatcaaggagagctagaaacttttagcttgttcaaccacgatgagatgcaagtcgttttctcaccagcaaccccctctgatcgcaactggacagatccttcttatcacctgcccgcattttacgagctatgggcacgctgggccgataacaacaatcaattttgggcagatctagccgtaaaaagtcgagaattttttaaagataccgcacacccagttaccggcttgtctccagattacgcttattttgatggctcgcctcatggcgactttcaacactggaaagacacattccagtacgatgcatggcgtaccattggcaatgctgcgatggatttttcttggtggcaaaacgatccttggcaaaccacttgggccaaccgactacaagctttctttgaaagtgaaggtgtggatgcttattccagcctatacgagctagacggcacgccttatgaaaataatgccgaccactcacctgggttagttgccatgaatgcggtagcaagcttagcggccgatgatgctcgggcctggaaatttgtgcaagctttgtggaacaccgaggtacccagtggtaagtggcgctattacgatggctcgctgtatatgcttggcatgcttgccgtaagcggtaagtatcatgtgatttgtcct

SEQ ID NO: 14 - protein, 550 aa, Marinimicrobium species
MRHIQKLLTVSLLSAVTHSAHAEILQHTELENTALLPGYTNIIDQPFLGIATYANGDGASAEAIVNTAPGTFRLDVRGASSASNSAGISAYLDTRRLGQLSFSGQTPSVQSLEFELDNNSSVEQIRLVLESDTGANDTLLDWFELHRIGDIPEPPPAPVLPSTGSYESGVYRNLFAELGYSNAEVDTKVQSAYDQLFHSPDQQNEALFVPVGSDMAYIWDVGNNDVRSEGMSYGMMMAVQMGRKDDFDRLWKWAHTYSLNKSGDMKGYFAWQVSTSGNVLDKNPAPDGEEYFVTALFFASHLWGDGNGIYDYSAQANSILDDLYSNGQTRYNNQGELETFSLFNHDEMQVVFSPATPSDRNWTDPSYHLPAFYELWARWADNNNQFWADLAVKSREFFKDTAHPVTGLSPDYAYFDGSPHGDFQHWKDTFQYDAWRTIGNAAMDFSWWQNDPWQTTWANRLQAFFESEGVDAYSSLYELDGTPYENNADHSPGLVAMNAVASLAADDARAWKFVQALWNTEVPSGKWRYYDGSLYMLGMLAVSGKYHVIC

SEQ ID NO: 15 - protein, 529 aa, Marinimicrobium species
EILQHTELENTALLPGYTNIIDQPFLGIATYANGDGASAEAIVNTAPGTFRLDVRGASSASNSAGISAYLDTRRLGQLSFSGQTPSVQSLEFELDNNSSVEQIRLVLESDTGANDTLLDWFELHRIGDIPEPPPAPVLPSTGSYESGVYRNLFAELGYSNAEVDTKVQSAYDQLFHSPDQQNEALFVPVGSDMAYIWDVGNNDVRSEGMSYGMMMAVQMGRKDDFDRLWKWAHTYSLNKSGDMKGYFAWQVSTSGNVLDKNPAPDGEEYFVTALFFASHLWGDGNGIYDYSAQANSILDDLYSNGQTRYNNQGELETFSLFNHDEMQVVFSPATPSDRNWTDPSYHLPAFYELWARWADNNNQFWADLAVKSREFFKDTAHPVTGLSPDYAYFDGSPHGDFQHWKDTFQYDAWRTIGNAAMDFSWWQNDPWQTTWANRLQAFFESEGVDAYSSLYELDGTPYENNADHSPGLVAMNAVASLAADDARAWKFVQALWNTEVPSGKWRYYDGSLYMLGMLAVSGKYHVICP

SEQ ID NO: 16 - DNA, 1149 bp, Artificial sequence
  Synthetic construct
  CDS (1)..(1146): GH8 xylanase, N-terminally truncated without signal or CBM.
gagggagcttattatacaggaatatacaggaatatgtttaaagaacttctcaataagtcggatgctgagatcaatacaaagatcaataacgcatttcagcagatcttccacggatcttctaatgagaagttatactatgaggtaggcaatgacatggcttatattctggatgtaaacaataacgatgtacgttcagaaggtatgtcttatggtatgatgatttgtgctcagcttgataaacaagtggaatttaataaaatatggaagtgggcaaagacctatatgcaatatggaaccaataataactttaatggctattttgcatggcagatgaataccaatggaacgattaagggaaacagcccggcatcagatggagaagcttattttattacagcgcttttcttcgcagcgaacagatggggaaatggagatggtatttttaattatgaagcagaagcgcaggatattctaaacaaggtgatgagtaaaaccggagcgggaagtgtttataaccttttcaataccaactccaagcttatcacatttgtaccttacggagattcttacaatttcacagacccgtcttataaccttccgggtttttgggaactttggagcagatggtccaagacaaatcagtctttctgggctcagacacctgcagcttcccgtaaattgcttcgtgatgcatcacattcatcatcgggtttaacaactgattattcaaattttgacggaacaccaaaagaagtttcatacaatacagatgcagacaggtttatgtatgatgcctggagaagcattatgaacctgagtgtggactatcattggtttaaggcagatgctcaacagccggtaatagctgaaaggtatttgacattcttcaaaaatcagggttccggctataaaaatcattacgattggaatggtgcaaatggaggaggggatcattcaacaggtctggtagcttgtaatgctgttgcctcacttgctgtaaataatacatcattgactacgccatttgtccaggaattgtggaatacagcaatccctacaggcacatacagatattatgacgggatgttgtatatgctcgggctattaaatgtttcaggtaatttcaaggtatggaaacctgcctgtcct

SEQ ID NO: 17 - protein, 382 aa, Artificial sequence
  Synthetic Construct
EGAYYTGIYRNMFKELLNKSDAEINTKINNAFQQIFHGSSNEKLYYEVGNDMAYILDVNNNDVRSEGMSYGMMICAQLDKQVEFNKIWKWAKTYMQYGTNNNFNGYFAWQMNTNGTIKGNSPASDGEAYFITALFFAANRWGNGDGIFNYEAEAQDILNKVMSKTGAGSVYNLFNTNSKLITFVPYGDSYNFTDPSYNLPGFWELWSRWSKTNQSFWAQTPAASRKLLRDASHSSSGLTTDYSNFDGTPKEVSYNTDADRFMYDAWRSIMNLSVDYHWFKADAQQPVIAERYLTFFKNQGSGYKNHYDWNGANGGGDHSTGLVACNAVASLAVNNTSLTTPFVQELWNTAIPTGTYRYYDGMLYMLGLLNVSGNFKVWKPAC

SEQ ID NO: 18 - protein, 383 aa, Artificial sequence
  Synthetic construct
EGAYYTGIYRNMFKELLNKSDAEINTKINNAFQQIFHGSSNEKLYYEVGNDMAYILDVNNNDVRSEGMSYGMMICAQLDKQVEFNKIWKWAKTYMQYGTNNNFNGYFAWQMNTNGTIKGNSPASDGEAYFITALFFAANRWGNGDGIFNYEAEAQDILNKVMSKTGAGSVYNLFNTNSKLITFVPYGDSYNFTDPSYNLPGFWELWSRWSKTNQSFWAQTPAASRKLLRDASHSSSGLTTDYSNFDGTPKEVSYNTDADRFMYDAWRSIMNLSVDYHWFKADAQQPVIAERYLTFFKNQGSGYKNHYDWNGANGGGDHSTGLVACNAVASLAVNNTSLTTPFVQELWNTAIPTGTYRYYDGMLYMLGLLNVSGNFKVWKPACP

SEQ ID NO: 19 - DNA, 1260 bp, Artificial sequence
  Synthetic construct
  CDS (1)..(1257): GH8 xylanase
  sig_peptide (1)..(84)
  mat_peptide (85)..(1257)
atgaagaatcttttccccaaaattaatttgcttggcagattactggtggtactgctggtttgcacatggagtagtagccatgcacaggatctctgtaatgaaaccggacagggagcctattttacaggtgtatacagaaacatgttcaaggaacttttgaacaagagtgatgcggaggtcaattccaagatcaatactgcattccagcagattttttatggtagttctaatcagaaattatattatgaggttgggcaggacatggcctatatccaggatatagccaataatgacgtccgctcagagggcatgtcttacgggctgatgatttgtgtacaactcgacaagcaggcagagtttaacaaactgtggaaatgggcaaaaacctacatgcagcacacttcaggaaatctgaacggttttttcaaatggcagttgaatacaaacgggaatgcaatagacaataatcctgctcctgacggagaagcatattttattactgccttattcttcgccgcaaaccgttggggaaatggaacaggaatttttaattatgaagccgaagctcaggatgctattagcaaggtaatgagcaaaacaggtgctggtggtataaacaatctgttcaatgccaattcaaaattaataacattcggaccaaattcaggctcatacgattatacagacccttcttacaatctgccaggattctgggaattgtgggctcgatggtcaaaaaccaatcaatcgttctggtctcagaccccaggggctgcccggaagttgcttcgcgatgcatctcattccagttcaggcctgacaactgactattcaaatttcgacggaacacctaaaactacatcattcaattccggtagccataggtttatgtatgatgcatggcgcagtataatgaacatgagtatggactaccactggttcaagaccgatgcccagcagcctgtgattgcagaacgttatcttaccttctttaagaaccagggctcaggttacaaaaatcattacgactggaatggctctaatggaggaggtgatcattctaccggtctggttgcctgcaatgcagttgcttcacttgcttgtgacaatacgtctcttacaacaccttttgtccaggagttgtggaatgtgggtgttccaagcggtacttacagatactatgatggtatgttgtacatgctttccttgttgaatgtatccggaaatttcaaggtatggaaaccagcttgtggaaatcct

SEQ ID NO: 20 - protein, 419 aa, Artificial sequence
  Synthetic Construct
MKNLFPKINLLGRLLVVLLVCTWSSSHAQDLCNETGQGAYFTGVYRNMFKELLNKSDAEVNSKINTAFQQIFYGSSNQKLYYEVGQDMAYIQDIANNDVRSEGMSYGLMICVQLDKQAEFNKLWKWAKTYMQHTSGNLNGFFKWQLNTNGNAIDNNPAPDGEAYFITALFFAANRWGNGTGIFNYEAEAQDAISKVMSKTGAGGINNLFNANSKLITFGPNSGSYDYTDPSYNLPGFWELWARWSKTNQSFWSQTPGAARKLLRDASHSSSGLTTDYSNFDGTPKTTSFNSGSHRFMYDAWRSIMNMSMDYHWFKTDAQQPVIAERYLTFFKNQGSGYKNHYDWNGSNGGGDHSTGLVACNAVASLACDNTSLTTPFVQELWNVGVPSGTYRYYDGMLYMLSLLNVSGNFKVWKPACGN

SEQ ID NO: 21 - protein, 392 aa, Artificial sequence
  Synthetic construct
QDLCNETGQGAYFTGVYRNMFKELLNKSDAEVNSKINTAFQQIFYGSSNQKLYYEVGQDMAYIQDIANNDVRSEGMSYGLMICVQLDKQAEFNKLWKWAKTYMQHTSGNLNGFFKWQLNTNGNAIDNNPAPDGEAYFITALFFAANRWGNGTGIFNYEAEAQDAISKVMSKTGAGGINNLFNANSKLITFGPNSGSYDYTDPSYNLPGFWELWARWSKTNQSFWSQTPGAARKLLRDASHSSSGLTTDYSNFDGTPKTTSFNSGSHRFMYDAWRSIMNMSMDYHWFKTDAQQPVIAERYLTFFKNQGSGYKNHYDWNGSNGGGDHSTGLVACNAVASLACDNTSLTTPFVQELWNVGVPSGTYRYYDGMLYMLSLLNVSGNFKVWKPACGNP

SEQ ID NO: 22 - DNA, 1332 bp, Artificial sequence
  Synthetic construct
  CDS (1)..(1329): GH8 xylanase
  sig_peptide (1)..(96)
  mat_peptide (97)..(1329)
atgtcagcacctaaatttttgctgttatacaaaccggcactactggtttgcagcctttgcctgatagctctgttagcagcctgtacggctaaccggcaaacagcaactatagtgcccgggcaggcagaactgggcagtgcttttagtggccgttacgctaacttatttgcgcagcagggctacagccagcaggccattaacgccaaaattagccaggcttttaaccagctgttttatggcgcgccagatagccaggcagtgtattttgctgatggcagtaacgggtatggcaccaaggcatatattgcagatattaataataatgatgtgcgctctgagggcatgtcgtacggcatgatgattgccgtgcagctgaataaacaggctgaatttaatgcgctgtggaactgggctgccagccatatgtaccatagcgatccggcgcatccggcttatggctactttgcctggtctgtaggtaccaacggtgtggcgctggatgaaatgcctgcgcctgatggcgaagaatattttgctacagcgctatattttgcttcggtacgctggggcgatggggctggcatatatcagtacaaggccaaagccgacgaattgctgcagcatatgcgccaccgcaacattattaccggccagaccttacgtggcagccaaaccggcaccaatttgtttcatcctgaatatgccatggtgcgctttacacccgatttagccaatgccgaccataccgatgcctcttaccacttaccggctttttatgaagtgtgggccagaatggggccagcagccgaccgggcattctggcaacaggccacccgggttagccgtgattattttagcaaggcagcgcacccgcaaaccggcctgacaccggattacggcaactttgacggcagcccctgggcagcgccctggcggccagagtcggccgattttcgctttgatgcctggcgcacggcgatgaactggtcatttgactggagctggtggcgcaaagatgccagagcggtagaactaagcaaccggctgcaggcgttttttatcagcgaaggcttaagcaactacagcagccaatacagcctggacggcaaaaagctggatcacacgcagcaaagtggtttagtggcaatgaatgccgtagccagcctggcagctacagggcccgatgccggtaaatttgtgcaggctttgtggcaactgccggtaccggaaggccactaccgttactatgacggcatgttgtatatgctggcattgttgcacgtaagcgggcaatttcaggcctggttgcctgcagcagataaaacagaataa

SEQ ID NO: 23 - protein, 443 aa, Artificial sequence
  Synthetic Construct
MSAPKFLLLYKPALLVCSLCLIALLAACTANRQTATIVPGQAELGSAFSGRYANLFAQQGYSQQAINAKISQAFNQLFYGAPDSQAVYFADGSNGYGTKAYIADINNNDVRSEGMSYGMMIAVQLNKQAEFNALWNWAASHMYHSDPAHPAYGYFAWSVGTNGVALDEMPAPDGEEYFATALYFASVRWGDGAGIYQYKAKADELLQHMRHRNIITGQTLRGSQTGTNLFHPEYAMVRFTPDLANADHTDASYHLPAFYEVWARMGPAADRAFWQQATRVSRDYFSKAAHPQTGLTPDYGNFDGSPWAAPWRPESADFRFDAWRTAMNWSFDWSWWRKDARAVELSNRLQAFFISEGLSNYSSQYSLDGKKLDHTQQSGLVAMNAVASLAATGPDAGKFVQALWQLPVPEGHYRYYDGMLYMLALLHVSGQFQAWLPAADKTE

SEQ ID NO: 24 - protein, 411 aa, Artificial sequence
  Synthetic construct
QTATIVPGQAELGSAFSGRYANLFAQQGYSQQAINAKISQAFNQLFYGAPDSQAVYFADGSNGYGTKAYIADINNNDVRSEGMSYGMMIAVQLNKQAEFNALWNWAASHMYHSDPAHPAYGYFAWSVGTNGVALDEMPAPDGEEYFATALYFASVRWGDGAGIYQYKAKADELLQHMRHRNIITGQTLRGSQTGTNLFHPEYAMVRFTPDLANADHTDASYHLPAFYEVWARMGPAADRAFWQQATRVSRDYFSKAAHPQTGLTPDYGNFDGSPWAAPWRPESADFRFDAWRTAMNWSFDWSWWRKDARAVELSNRLQAFFISEGLSNYSSQYSLDGKKLDHTQQSGLVAMNAVASLAATGPDAGKFVQALWQLPVPEGHYRYYDGMLYMLALLHVSGQFQAWLPAADKTE

SEQ ID NO: 25 - protein, 27 aa, Bacillus clausii
MKKPLGKIVASTALLISVAFSSSIASA

SEQ ID NO: 26 - protein, 5 aa, Artificial sequence
  Synthetic construct
  MISC_FEATURE (1)..(1): X is T or S in position 1
  MISC_FEATURE (3)..(3): X is P or A in position 3
XDXSY

SEQ ID NO: 27 - protein, 7 aa, Artificial sequence
  Synthetic construct
  MISC_FEATURE (3)..(3): X is F, Y, V, I, L or M in position 3.
  MISC_FEATURE (4)..(4): X is G or S in position 4.
MNXXMDY

SEQ ID NO: 28 - protein, 5 aa, Artificial sequence
  Snythetic construct
  MISC_FEATURE (2)..(2): X is I or L in position 2
AXWNW

SEQ ID NO: 29 - protein, 6 aa, Artificial sequence
  Synthetic construct
  MISC_FEATURE (2)..(2): X is I or F in position 2.
WXAAAL

SEQ ID NO: 30 - protein, 4 aa, Artificial sequence
  Synthetic construct
DEAG

SEQ ID NO: 31 - protein, 7 aa, Artificial sequence
  Synthetic construct
AANAGGA

(19) United States
(12) Patent Application Publication
(10) Pub. No.: US 2023/0183385 A1
(43) Pub. Date: Jun. 15, 2023

(54) Wheat Milling Process and GH8 Xylanases

(71) Applicant: Novozymes A/S, Bagsvaerd (DK)

(72) Inventors: James Lavigne, Wake Forest (NC); Gernot Abel, Kobenhavn (DK); Jesper Salomon, Holte (DK); Tucker Shepperd, Franklinton, NC (US)

(73) Assignee: Novozymes A/S, Bagsvaerd (DK)

(21) Appl. No.: 16/954,281

(22) PCT Filed: Dec. 20, 2018
(86) PCT No.: PCT/EP2018/086117

§ 371 Date: Jun. 16, 2020

Related U.S. Application Data
(60) Provisional application No. 62/609,409, filed on Dec. 22, 2017.

(51) Int. Cl. C08B 30/04; C12N 9/24

(52) U.S. Cl. CPC C08B 30/046; C12N 9/248; C12Y 302/01008

(57) ABSTRACT

The present invention relates to processes for separating wheat flour into two or more fractions including a gluten fraction and a starch fraction, comprising the steps of: a) mixing wheat flour and water; b) adding one or more polypeptide (s) having GH8 xylanase activity; c) incubating the mixture for a predefined period of time; d) separating the mixture into two or more fractions including a gluten rich fraction and a starch rich fraction; and e) recovering the two or more fractions including a gluten rich fraction and a starch rich fraction.

### REFERENCE TO SEQUENCE LISTING

This application contains a Sequence Listing in computer readable form. The computer readable form is incorporated herein by reference.

### FIELD OF THE INVENTION

The present invention relates to an improved process of treating crop kernels to provide a starch product of high quality suitable for conversion of starch into mono- and oligosaccharides, ethanol, sweeteners, etc. Further, the invention also relates to an enzyme composition comprising one or more enzyme activities suitable for the process of the invention and to the use of the composition of the invention.

### BACKGROUND OF THE INVENTION

Before starch, which is an important constituent in the kernels of most crops, such as corn, wheat, rice, sorghum bean, barley or fruit hulls, can be used for conversion of starch into saccharides, such as dextrose, fructose; alcohols, such as ethanol; and sweeteners, the starch must be made available and treated in a manner to provide a high purity starch. If starch contains more than 0.5% impurities, including the proteins, it is not suitable as starting material for starch conversion processes. To provide such pure and high quality starch product starting out from the kernels of crops, the kernels are often milled, as will be described further below.

Wet milling is often used for separating crop kernels into its four basic components: starch, germ, fiber and protein, all of which are valuable.

Separation of wheat flour into two or more fraction including a gluten fraction and a starch fraction is a well, known industrial process and in general it is performed using a process containing the steps of
a) Mixing water and wheat flour;
b) Incubating the mixture in a period for allow gluten to form a gluten network;
c) Separating the mixture into at least two fractions, a gluten rich fraction and a starch rich fraction; and
d) Optional further purifications of the fractions.

Several different enzymes have been suggested for the crop kernel steeping and/or wet milling processes. However, there remains a need for improving wet-milling processes to achieve, e.g., higher protein and starch yields, lower process flow viscosity etc.

### SUMMARY OF THE INVENTION

The inventors tested eight glycosyl hydrolase family 8 (GH8) xylanases for their ability to lower the viscosity of a wheat flour slurry, representative for the typical product flow from a crop kernel wet-milling process, and found to their surprise that all eight GH8 xylanases were able to lower the viscosity of the slurry significantly (FIG. 4).

Accordingly, In a first aspect, the invention relates to a process for separating wheat flour into two or more fraction including a gluten fraction and a starch fraction, comprising the steps of:
a) making a mixture of wheat flour and water;
b) adding one or more polypeptide (s) having GH8 Xylanase activity;
c) incubating the mixture for a predefined period of time;
d) separating the mixture into two or more fractions including a gluten fraction and a starch fraction using a number of sifting and centrifugation steps; and recovering the two or more fractions including a gluten fraction and a starch fraction.

In a second aspect, the invention relates to an enzyme composition comprising a polypeptide having GH8 xylanase activity, wherein

the polypeptide having GH8 xylanase activity is a member of the DPSY Glade as defined herein; preferably the polypeptide having GH8 xylanase activity is a member of at least one of the following clades as defined herein: the SMDY Glade, the ALWNW Glade, the WFAAAL Glade, and the DEAG Glade.
a) In a final aspect, the invention relates to the use of a polypeptide having GH8 xylanase activity in a process for treating crop kernels,
b) making a mixture of wheat flour and water;
c) adding one or more polypeptide (s) having GH8 Xylanase activity;
d) incubating the mixture for a predefined period of time;
e) separating the mixture into two or more fractions including a gluten fraction and a starch fraction using a number of sifting and centrifugation steps; and

recovering the two or more fractions including a gluten fraction and a starch fraction.

### DRAWINGS

FIG. 1 shows how the GH8 xylanase polypeptides can be separated into multiple distinct clades, where each clade was named based on its conserved motif.

FIG. 2 shows a phylogenetic tree of the xylanase polypeptides of the invention.

FIG. 3 shows an alignment of the GH8 xylanases tested herein.

FIG. 4 shows that eight GH8 xylanase clade “DPSY” members, as defined above, are effective at reducing the viscosity of the wheat slurry in the ViPr assay.

FIG. 5 shows that the wheat slurry viscosity reductions by GH8 xylanases are better than that of a commercially available GH10 xylanase product (Shearzyme®, Novozymes).

FIG. 6 shows that the Bacillus sp. KK-1 wildtype GH8 xylanase reduced the viscosity of a wheat slurry about 4-fold compared to no enzyme.

FIG. 7 shows that the Bacillus sp. KK-1 wildtype GH8 xylanase improved the protein recovery from about 5% to 25-30%, i.e., close to a 6-fold improvement.

### DEFINITIONS

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

cDNA: The term “cDNA” means a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant polynucleotide.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of the polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to additional nucleotides that provide for its expression.

Fragment: The term “fragment” means a polypeptide having one or more (e.g. several) amino acids deleted from the amino and/or carboxyl terminus of a mature polypeptide; wherein the fragment has protease activity. In one aspect, a fragment contains at least 330 amino acid residues (e.g., amino acids 20 to 349 of SEQ ID NO: 2); in another aspect a fragment contains at least 345 amino acid residues (e.g., amino acids 10 to 354 of SEQ ID NO: 2); in a further aspect a fragment contains at least 355 amino acid residues (e.g., amino acids 5 to 359 of SEQ ID NO: 2).

Host cell: The term “host cell” means any cell type that is susceptible to transformation, transfection, transduction, and the like with a nucleic acid construct or expression vector comprising a polynucleotide of the present invention. The term “host cell” encompasses any progeny of a parent cell that is not identical to the parent cell due to mutations that occur during replication.

Isolated polynucleotide: The term “isolated polynucleotide” means a polynucleotide that is modified by the hand of man relative to that polynucleotide as found in nature. In one aspect, the isolated polynucleotide is at least 1% pure, e.g., at least 5% pure, more at least 10% pure, at least 20% pure, at least 40% pure, at least 60% pure, at least 80% pure, at least 90% pure, and at least 95% pure, as determined by agarose electrophoresis. The polynucleotides may be of genomic, cDNA, RNA, semisynthetic, synthetic origin, or any combinations thereof.

Isolated polypeptide: The term “isolated polypeptide” means a polypeptide that is modified by the hand of man relative to that polypeptide as found in nature. In one aspect, the polypeptide is at least 1% pure, e.g., at least 5% pure, at least 10% pure, at least 20% pure, at least 40% pure, at least 60% pure, at least 80% pure, and at least 90% pure, as determined by SDS-PAGE.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 366 in the numbering of SEQ ID NO: 2 based on sequencing using Edman degredation and intact molecular weight analysis of the mature polypeptide with N-terminal HQ-tag. Using the prediction program SignalP (Nielsen et al., 1997, Protein Engineering 10: 1-6), amino acids −27 to −1 in the numbering of SEQ ID NO:2 are predicted to be the signal peptide. It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having protease activity. In one aspect, the mature polypeptide coding sequence is nucleotides 82 to 1302 in the numbering of SEQ ID NO:1 based on the determination of the mature polypeptide by Edman degradation and intact molecular weight analysis of the mature polypeptide with N-terminal HQ-tag. Furthermore nucleotides 1 to 81 in the numbering of SEQ ID NO:1 are predicted to encode a signal peptide based on the prediction program SignalP (Nielsen et al., 1997, Protein Engineering 10: 1-6).

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

Operably linked: The term “operably linked” means a configuration in which a control sequence is placed at an appropriate position relative to the coding sequence of a polynucleotide such that the control sequence directs the expression of the coding sequence.

Protease activity: The term “protease activity” means proteolytic activity (EC 3.4). There are several protease activity types such as trypsin-like proteases cleaving at the carboxyterminal side of Arg and Lys residues and chymotrypsin-like proteases cleaving at the carboxyterminal side of hydrophobic amino acid residues. Proteases of the invention are serine endopeptidases (EC 3.4.21) with acidic pH-optimum (pH optimum<pH 7).

Protease activity can be measured using any assay, in which a substrate is employed, that includes peptide bonds relevant for the specificity of the protease in question. Assay-pH and assay-temperature are likewise to be adapted to the protease in question. Examples of assay-pH-values are pH 2, 3, 4, 5, 6, 7, 8, 9, 10, 11, or 12. Examples of assay-temperatures are 15, 20, 25, 30, 35, 37, 40, 45, 50, 55, 60, 65, 70, 80, 90, or 95° C. Examples of general protease substrates are casein, bovine serum albumin and haemoglobin. In the classical Anson and Mirsky method, denatured haemoglobin is used as substrate and after the assay incubation with the protease in question, the amount of trichloroacetic acid soluble haemoglobin is determined as a measurement of protease activity (Anson, M. L. and Mirsky, A. E., 1932, J. Gen. Physiol. 16: 59 and Anson, M. L., 1938, J. Gen. Physiol. 22: 79).

Sequence Identity: The relatedness between two amino acid sequences or between two nucleotide sequences is described by the parameter “sequence identity”.

For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. Version 6.1.0 was used. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. Version 6.1.0 was used. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Stringency conditions: The different strigency conditions are defined as follows.

The term “very low stringency conditions” means for probes of at least 100 nucleotides in length, prehybridization and hybridization at 42° C. in 5× SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2× SSC, 0.2% SDS at 45° C.

The term “low stringency conditions” means for probes of at least 100 nucleotides in length, prehybridization and hybridization at 42° C. in 5× SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2× SSC, 0.2% SDS at 50° C.

The term “medium stringency conditions” means for probes of at least 100 nucleotides in length, prehybridization and hybridization at 42° C. in 5× SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2× SSC, 0.2% SDS at 55° C.

The term “medium-high stringency conditions” means for probes of at least 100 nucleotides in length, prehybridization and hybridization at 42° C. in 5× SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2× SSC, 0.2% SDS at 60° C.

The term “high stringency conditions” means for probes of at least 100 nucleotides in length, prehybridization and hybridization at 42° C. in 5× SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2× SSC, 0.2% SDS at 65° C.

The term “very high stringency conditions” means for probes of at least 100 nucleotides in length, prehybridization and hybridization at 42° C. in 5× SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2× SSC, 0.2% SDS at 70° C.

Subsequence: The term “subsequence” means a polynucleotide having one or more (several) nucleotides deleted from the 5′ and/or 3′ end of a mature polypeptide coding sequence; wherein the subsequence encodes a fragment having protease activity. In one aspect, a subsequence contains at least 990 nucleotides (e.g., nucleotides 139 to 1128 of SEQ ID NO: 1), e.g., and at least 1035 nucleotides (e.g., nucleotides 109 to 1143 of SEQ ID NO: 1); e.g., and at least 1065 nucleotides (e.g., nucleotides 94 to 1158 of SEQ ID NO: 1).

Substantially pure polynucleotide: The term “substantially pure polynucleotide” means a polynucleotide preparation free of other extraneous or unwanted nucleotides and in a form suitable for use within genetically engineered polypeptide production systems. Thus, a substantially pure polynucleotide contains at most 10%, at most 8%, at most 6%, at most 5%, at most 4%, at most 3%, at most 2%, at most 1%, and at most 0.5% by weight of other polynucleotide material with which it is natively or recombinantly associated. A substantially pure polynucleotide may, however, include naturally occurring 5′ and 3′ untranslated regions, such as promoters and terminators. Preferably, the polynucleotide is at least 90% pure, e.g., at least 92% pure, at least 94% pure, at least 95% pure, at least 96% pure, at least 97% pure, at least 98% pure, at least 99% pure, and at least 99.5% pure by weight. The polynucleotides of the present invention are preferably in a substantially pure form.

Substantially pure polypeptide: The term “substantially pure polypeptide” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the polypeptide is at least 92% pure, e.g., at least 94% pure, at least 95% pure, at least 96% pure, at least 97% pure, at least 98% pure, at least 99%, at least 99.5% pure, and 100% pure by weight of the total polypeptide material present in the preparation. The polypeptides of the present invention are preferably in a substantially pure form. This can be accomplished, for example, by preparing the polypeptide by well-known recombinant methods or by classical purification methods.

Variant: The term “variant” means a polypeptide having protease activity comprising an alteration, i.e., a substitution, insertion, and/or deletion of one or more (several) amino acid residues at one or more (several) positions. A substitution means a replacement of an amino acid occupying a position with a different amino acid; a deletion means removal of an amino acid occupying a position; and an insertion means adding 1-3 amino acids adjacent to an amino acid occupying a position. The variants of the present invention have at least 20%, e.g., at least 40%, at least 50%, at least 60%, at least 70%, at least 80%, at least 90%, at least 95%, or at least 100% of the protease activity of the polypeptide of SEQ ID NO: 5, SEQ ID NO: 6, or the mature polypeptide of SEQ ID NO: 2 or SEQ ID NO: 4.

In one aspect, the variant differs by up to 10 amino acids, e.g., 1, 2, 3, 4, 5, 6, 7, 8, 9, or 10, from the mature polypeptide of a SEQ ID NO: as identified herein. In another embodiment, the present invention relates to variants of the mature polypeptide of a SEQ ID NO: herein comprising a substitution, deletion, and/or insertion at one or more (e.g., several) positions. In an embodiment, the number of amino acid substitutions, deletions and/or insertions introduced into the mature polypeptide of a SEQ ID NO: herein is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. For purposes of the present invention, beta-glucosidase activity is determined using p-nitrophenyl-beta-D-glucopyranoside as substrate according to the procedure of Venturi et al., 2002, Extracellular beta-D-glucosidase from Chaetomium thermophilum var. coprophilum: production, purification and some biochemical properties, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. For purposes of the present invention, one unit of beta-xylosidase is defined as 1.0 μmole of p-nitrophenolate anion produced per minute at 40° C., pH 5 from 1 mM p-nitrophenyl-beta-D-xyloside as substrate in 100 mM sodium citrate containing 0.01% TWEEN® 20.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain (Teeri, 1997, Crystalline cellulose degradation: New insight into the function of cellobiohydrolases, Trends in Biotechnology 15: 160-167; Teeri et al., 1998, Trichoderma reesei cellobiohydrolases: why so efficient on crystalline cellulose?, Biochem. Soc. Trans. 26: 173-178). Cellobiohydrolase activity is determined according to the procedures described by Lever et al., 1972, Anal. Biochem. 47: 273-279; van Tilbeurgh et al., 1982, FEBS Letters, 149: 152-156; van Tilbeurgh and Claeyssens, 1985, FEBS Letters, 187: 283-288; and Tomme et al., 1988, Eur. J. Biochem. 170: 575-581. In the present invention, the Tomme et al. method can be used to determine cellobiohydrolase activity.

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Measurement of cellulase activities, Pure Appl. Chem. 59: 257-68).

Cellulosic material: The term “cellulosic material” means any material containing cellulose. Cellulose is a homopolymer of anyhdrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Endoglucanase: The term “endoglucanase” means an endo-1,4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Family 61 glycoside hydrolase: The term “Family 61 glycoside hydrolase” or “Family GH61” or “GH61” means a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696. The enzymes in this family were originally classified as a glycoside hydrolase family based on measurement of very weak endo-1,4-beta-D-glucanase activity in one family member. The structure and mode of action of these enzymes are non-canonical and they cannot be considered as bona fide glycosidases. However, they are kept in the CAZy classification on the basis of their capacity to enhance the breakdown of lignocellulose when used in conjunction with a cellulase or a mixture of cellulases.

Hemicellulolytic enzyme or hemicellulase: The term “hemicellulolytic enzyme” or “hemicellulase” means one or more (e.g., several) enzymes that hydrolyze a hemicellulosic material. See, for example, Shallom, D. and Shoham, Y. Microbial hemicellulases. Current Opinion In Microbiology, 2003, 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates of these enzymes, the hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) data-base. Hemicellulolytic enzyme activities can be measured according to Ghose and Bisaria, 1987, Pure & Appl. Chem. 59: 1739-1752, at a suitable temperature, e.g., 50° C., 55° C., or 60° C., and pH, e.g., 5.0 or 5.5.

Polypeptide having cellulolytic enhancing activity: The term “polypeptide having cellulolytic enhancing activity” means a GH61 polypeptide that catalyzes the enhancement of the hydrolysis of a cellulosic material by enzyme having cellulolytic activity. In one aspect, a mixture of CELLUCLAST® 1.5 L (Novozymes NS, Bagsvrd, Denmark) in the presence of 2-3% of total protein weight Aspergillus oryzae beta-glucosidase (recombinantly produced in Aspergillus oryzae according to WO 02/095014) or 2-3% of total protein weight Aspergillus fumigatus beta-glucosidase (recombinantly produced in Aspergillus oryzae as described in WO 2002/095014) of cellulase protein loading is used as the source of the cellulolytic activity.

The GH61 polypeptides having cellulolytic enhancing activity enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. For purposes of the present invention, xylanase activity is determined with 0.2% AZCL-arabinoxylan as substrate in 0.01% TRITON® X-100 and 200 mM sodium phosphate buffer pH 6 at 37° C. One unit of xylanase activity is defined as 1.0 micromole of azurine produced per minute at 37° C., pH 6 from 0.2% AZCL-arabinoxylan as substrate in 200 mM sodium phosphate pH 6 buffer.

Crop kernels: The term “crop kernels” includes kernels from, e.g., corn (maize), rice, barley, sorghum bean, fruit hulls, and wheat. Corn kernels are exemplary. A variety of corn kernels are known, including, e.g., dent corn, flint corn, pod corn, striped maize, sweet corn, waxy corn and the like.

In an embodiment, the corn kernel is yellow dent corn kernel. Yellow dent corn kernel has an outer covering referred to as the “Pericarp” that protects the germ in the kernels. It resists water and water vapour and is undesirable to insects and microorganisms.

The only area of the kernels not covered by the “Pericarp” is the “Tip Cap”, which is the attachment point of the kernel to the cob.

Germ: The “germ” is the only living part of the corn kernel. It contains the essential genetic information, enzymes, vitamins, and minerals for the kernel to grow into a corn plant. In yellow dent corn, about 25 percent of the germ is corn oil. The endosperm covered surrounded by the germ comprises about 82 percent of the kernel dry weight and is the source of energy (starch) and protein for the germinating seed. There are two types of endosperm, soft and hard. In the hard endosperm, starch is packed tightly together. In the soft endosperm, the starch is loose.

Starch: The term “starch” means any material comprised of complex polysaccharides of plants, composed of glucose units that occurs widely in plant tissues in the form of storage granules, consisting of amylose and amylopectin, and represented as (C6H10O5)n, where n is any number.

Milled: The term “milled” refers to plant material which has been broken down into smaller particles, e.g., by crushing, fractionating, grinding, pulverizing, etc.

Grind or grinding: The term “grinding” means any process that breaks the pericarp and opens the crop kernel.

Steep or steeping: The term “steeping” means soaking the crop kernel with water and optionally SO₂.

Dry solids: The term “dry solids” is the total solids of a slurry in percent on a dry weight basis.

Oligosaccharide: The term “oligosaccharide” is a compound having 2 to 10 monosaccharide units.

Wet milling benefit: The term “wet milling benefit” means one or more of improved starch yield and/or purity, improved gluten yield and/or purity, improved fiber purity, or steep water filtration, dewatering and evaporation, easier germ separation and/or better post-saccharification filtration, and process energy savings thereof.

### DETAILED DESCRIPTION OF THE INVENTION

Wheat Gluten Starch Separation

The invention relates to a method for separating wheat flour into two or more fractions including a gluten fraction and a starch fraction, comprising the steps of:
a) mixing wheat flour and water;
b) adding one or more polypeptide (s) having GH8 xylanase activity;
c) incubating the mixture for a predefined period of time;
d) separating the mixture into two or more fractions including a gluten rich fraction and a starch rich fraction; and
e) recovering the two or more fractions including a gluten rich fraction and a starch rich fraction;

wherein the one or more polypeptide(s) having GHG8 xylanase activity is (are) selected among polypeptides having lipase activity and having a sequence identity to one of SEQ ID NO: 2 of at least 60%.

The wheat flour may in principle be any wheat flour and the invention is not limited to any particular wheat variety, brand or milling procedure as known in the art.

Mixing wheat flour and water is the first step in the method of the invention and has the purpose of enable wheat flour hydration and gluten agglomeration through efficient mixing. This step is well known in the art and is sometimes also called Dough preparation. The step is performed by mixing water and wheat flour under agitation, forming a mixture or dough.

The amount of water added to the wheat flour depends on factors such as the particular process conditions, the particular wheat and the wheat variety used and will readily be determined by the person skilled in the art. Typically the amount of water added is in the range of 0.1-3 Liter per kg wheat flour, preferably 0.5-2.5 Liter per kg wheat flour, preferably 1-2 Liter per kg wheat flour. The condition such as pH and temperature is typically determined by the ingredients, meaning that the mixing is typically done without any adjustment of pH and temperature, so the pH and temperature is determined by the used raw materials.

According to the invention one or more polypeptides having GH8 xylanase activity is added to the mixture. The one or more polypeptides having GH8 xylanase activity may be added together with the wheat flour or it may be added after the wheat flour and water has been mixed. When the one or more polypeptides having GH8 xylanase activity has been added mixing should continue at least for a sufficient period to secure even distribution in the mixture or dough. The one or more polypeptides having GH8 xylanase activity is typically added in amounts in the rage of 0.1-500 μg enzyme protein per gram wheat flour (μg EP/g wheat), e.g. in the range of 1-200 μg EP/g wheat, e.g. in the range of 5-100 μg EP/g wheat.

In some embodiments one or more additional enzymes are added together with the one or more polypeptides having GH8 xylanase activity. In this connection “added together” is intended to mean that the one or more additional enzymes are added simultaneously or sequentially with the one or more polypeptide having GH8 xylanase activity so that both the one or more additional enzymes and the one or more polypeptides having GH8 xylanase activity are mixed and evenly distributed in the mixture or dough when the mixing process is completed. Thus, the one and more polypeptides having GH8 xylanase activity and the one or more additional enzymes may be added as a single composition or as two or more separate compositions each comprising one or more enzymes.

The one or more additional enzymes may be selected among cellulases, xylanases, proteases amylases, lipases and arabinofuranosidases

In a preferred embodiment a polypeptide having xylanase activity is added together with the polypeptide having lipase activity. The polypeptide having xylanase activity may be selected among GH10 or GH11 xylanases.

A preferred xylanase according to the invention is the GH10 xylanase disclosed in WO 97/021785.

A preferred lipase according to the invention is a lipase disclosed in PCTR/CN2018/116692, incorporated herein by reference.

Incubating the mixture for a predefined period of time. When the mixing is complete the mixture or dough is incubated in a predefined period to allow the gluten to form gluten network. Further the one or more polypeptides having lipase activity will during this period hydrolyse the lipids in the mixture or dough and the optional additional enzymes may act upon their substrates during this incubation period. This is also called dough maturation and is typically done in a maturation tank. Typically, the incubation is done at ambient temperature i.e. without temperature regulation. Thus the incubation typically takes place at a temperature in the range of 5-50° C., preferably in the range of 15-40° C. and most preferred in the range of 20-35° C.

The incubation is performed for a sufficient time to allow the gluten network to form and the duration is easily determined by the person skilled in the art. The mixture may be performed for a period in the range of 5 minutes to 8 hours, e.g. in the range of 15 minutes to 4 hours

Separating the mixture into two or more fractions including a gluten rich fraction and a starch rich fraction. After the incubation period the mixture is separated into two or more fractions including a starch rich fraction and a gluten rich fraction.

A starch rich fraction is in this application intended to mean a fraction that comprises at least 50% (w/w) starch, preferably at least 60% (w/w) starch, preferably at least 70% (w/w) starch, preferably at least 80% (w/w) starch, preferably at least 90% (w/w) starch, calculated based on the dry matter of the fraction.

A gluten rich fraction is in this application intended to mean a fraction that comprises at least 50% (w/w) gluten, preferably at least 60% (w/w) gluten, preferably at least 70% (w/w) gluten, preferably at least 80% (w/w) gluten, preferably at least 90% (w/w) gluten, calculated based on the dry matter of the fraction.

The separation step may be performed based on differences in solubility and density using methods and equipment known in the art.

In a preferred embodiment the separation step is performed using a 3 phase separator process separating the mixture or dough into a starch rich fraction; a gluten rich fraction; and a pentosan fraction having a high content of fibers, in particular pentosans such as arabinoxylans.

After the separation step separating the mixture/dough into two or more fractions including a gluten rich fraction and a starch rich fraction, each of these fractions may be subjected to additional separation steps in order to purify the fractions even further and avoid loss. Such operations are known in the art and are e.g. known as gluten washing, starch washing and fiber washing and are typically performed using a number of decanters, sedicanters, centrifuges, screens, hydrocyclones etc. as known in the art.

The separation steps have been completed and the two or more fractions have obtained their intended purity the fraction is recovered, typically by removing excess water and obtaining the fractions in dry stable form. Alternatively, the obtained fractions may immediately be further processed without drying.

In a further aspect the invention relates to the use of one or more polypeptides having GH8 xylanase activity, wherein the polypeptide having GH8 xylanase activity is a member of the DPSY clade as defined herein; preferably the polypeptide having GH8 xylanase activity is a member of at least one of the the following clades as defined herein: the SMDY clade, the ALWNW clade, the WFAAAL clade, and the DEAG clade.

There are several technical benefits to be derived from the process of the invention, including, an improved separation; preferably the process provides a reduced viscosity in the wheat flour slurry as determined herein and/or a higher protein recovery as determined herein. This has been reflected in that the capacity in the first separation step separating the mixture or dough into two or more fractions including a starch rich fraction and a gluten rich fraction compared with same.

Polypeptides Having GH8 xylanase Activity

GH8 Xylanase

Glycoside hydrolases E.C. 3.2.1. are a widespread group of enzymes that hydrolyse the glycosidic bond between two or more carbohydrates, or between a carbohydrate and a non-carbohydrate moiety. A classification system for glycoside hydrolases, based on sequence similarity, has led to the definition of >100 different families. This classification is available on the CAZy (http://www.cazy.org/GH1.html) web site and also discussed at CAZypedia, an online encyclopedia of carbohydrate active enzymes.

Glycoside hydrolase family 8 in CAZY, GH8, comprises inverting enzymes with several known activities, incl. chitosanase (EC 3.2.1.132); cellulase (EC 3.2.1.4); licheninase (EC 3.2.1.73); endo-1,4-β-xylanase (EC 3.2.1.8); reducing-end-xylose releasing exo-oligoxylanase (EC 3.2.1.156).

The second aspect of the invention relates to an enzyme composition comprising a polypeptide having GH8 xylanase activity, wherein the polypeptide having GH8 xylanase activity is a member of the DPSY clade as defined herein; preferably the polypeptide having GH8 xylanase activity is a member of at least one of the the following clades as defined herein: the SMDY clade, the ALWNW clade, the WFAAAL clade, and the DEAG clade.

In a preferred embodiment, the polypeptide having GH8 xylanase activity is selected from the group consisting of:
A. a polypeptide having at least 80%, at least 85%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 5, 11, 14, 17, 20 or 23; or the mature polypeptide of SEQ ID NO: 6, 12, 15, 18, 21 or 24;
B. a polypeptide encoded by a polynucleotide that hybridizes under high stringency conditions, or very high stringency conditions with
   (i) the polypeptide coding sequence of SEQ ID NO: 4, 10, 13, 16, 19, or 22;
   (ii) the mature polypeptide coding sequence of SEQ ID NO: 4, 10, 13, 16, 19, or 22,
   (iii) the full-length complementary strand of (i) or (ii);
C. a polypeptide encoded by a polynucleotide having at least 80%,at least 85%, at least 90%, at least 91%, at least 92%, at least 93%, at least 94%, at least 95%, at least 96%, at least 97%, at least 98%, at least 99% or at least 100% sequence identity to the mature polypeptide coding sequence of SEQ ID NO: 4, 10, 13, 16, 19, or 22;
D. a variant of the polypeptide of SEQ ID NO: 5, 11, 14, 17, 20 or 23; or the mature polypeptide of SEQ ID NO: 6, 12, 15, 18, 21 or 24 comprising a substitution, deletion, and/or insertion at one or more (several) positions; and
E. a fragment of a polypeptide of A., B., C. or D. having GH8 xylanase activity.

In another preferred embodiment, the polypeptide having GH8 xylanase activity is a polypeptide having at least 85%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 5, 11, 14, 17, 20 or 23; or the mature polypeptide of SEQ ID NO: 6, 12, 15, 18, 21 or 24.

Another preferred embodiment of the second aspect relates to, wherein the polypeptide having GH8 xylanase activity is a polypeptide encoded by a polynucleotide having at least 85%, at least 90%, at least 91%, at least 92%, at least 93%, at least 94%, at least 95%, at least 96%, at least 97%, at least 98%, at least 99% or at least 100% sequence identity to the mature polypeptide coding sequence of SEQ ID NO: 4, 10, 13, 16, 19, or 22.

Yet another preferred embodiment of the second aspect relates to, wherein the polypeptide having GH8 xylanase activity comprises or consists of SEQ ID NO: 5, 11, 14, 17, 20 or 23; or the mature polypeptide of SEQ ID NO: 6, 12, 15, 18, 21 or 24.

Preferably, the composition of the second aspect further comprises one or more enzyme selected from the group consisting of a beta-xylosidase, cellulase, hemi-celluase, lipase, endoglucanase, acetylan esterase, cellobiohydrolase I, cellobiohydrolase II, and GH61 polypeptide.

In an embodiment the polypeptide having GH8 xylanase activity used in the process of the invention has xylanase protease activity and are encoded by polynucleotides that hybridize under high stringency conditions, or very high stringency conditions with (i) the polypeptide coding sequence of SEQ ID NO: 4, 10, 13, 16, 19, or 22; (ii) the mature polypeptide coding sequence of SEQ ID NO: 4, 10, 13, 16, 19, or 22, (iii) the full-length complementary strand of (i) or (ii). (J. Sambrook, E. F. Fritsch, and T. Maniatis, 1989, Molecular Cloning, A Laboratory Manual, 2d edition, Cold Spring Harbor, N.Y.

The full-length or the mature polypeptide coding sequence of SEQ ID NO: 4, 10, 13, 16, 19, or 22, or a subsequence thereof, as well as the amino acid sequence of SEQ ID NO: 5, 11, 14, 17, 20 or 23; or the mature polypeptide of SEQ ID NO: 6, 12, 15, 18, 21 or 24, or a fragment thereof, may be used to design nucleic acid probes to identify and clone DNA encoding polypeptides having protease activity from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having protease activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with the full-length or the mature polypeptide coding sequence of SEQ ID NO: 4, 10, 13, 16, 19, or 22 or a subsequence thereof, the carrier material is preferably used in a Southern blot.

For one purpose of the present invention, hybridization indicates that the polynucleotide hybridizes to a labeled nucleic acid probe corresponding to the full-length or the mature polypeptide coding sequence of SEQ ID NO: 4, 10, 13, 16, 19, or 22, its complementary strand or a subsequence thereof under high to very high stringency conditions. Molecules to which the nucleic acid probe hybridizes under these conditions can be detected using, for example, X-ray film or any other detection means known in the art.

For long probes of at least 100 nucleotides in length, high to very high stringency conditions are defined as prehybridization and hybridization at 42° C. in 5× SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. The carrier material is finally washed three times each for 15 minutes using 2× SSC, 0.2% SDS at 65° C. (high stringency), and at 70° C. (very high stringency).

For short probes of about 15 nucleotides to about 70 nucleotides in length, stringency conditions are defined as prehybridization and hybridization at about 5° C. to about 10° C. below the calculated Tₘ using the calculation according to Bolton and McCarthy (1962, Proc. Natl. Acad. Sci. USA 48:1390) in 0.9 M NaCl, 0.09 M Tris-HCl pH 7.6, 6 mM EDTA, 0.5% NP-40, 1× Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally. The carrier material is finally washed once in 6× SCC plus 0.1% SDS for 15 minutes and twice each for 15 minutes using 6× SSC at 5° C. to 10° C. below the calculated Tₘ.

In another embodiment, the present invention relates to using variants comprising a substitution, deletion, and/or insertion at one or more (or several) positions of the mature polypeptide of SEQ ID NO: 6, 12, 15, 18, 21 or 24, or a homologous sequence. The amino acid changes may be of a minor nature, that is conservative amino acid substitutions, insertions or deletions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tag or HQ-tag, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges that are expected not to alter the specific activity substantially are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for protease activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with polypeptides that are related to the parent polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The total number of amino acid substitutions, deletions and/or insertions of the mature polypeptide of SEQ ID NO: 6, 12, 15, 18, 21 or 24 is not more than 20, e.g., 1, 2, 3, 4, 5, 6, 7, 8, 9, 10, 11, 12, 13, 14, 15, 16, 17, 18, 19 or 20.

The polypeptide may be hybrid polypeptide in which a portion of one polypeptide is fused at the N-terminus or the C-terminus of a portion of another polypeptide.

The polypeptide may be a fusion polypeptide or cleavable fusion polypeptide in which another polypeptide is fused at the N-terminus or the C-terminus of the polypeptide of the present invention. A fused polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The polypeptide may be expressed by a recombinant DNA sequence containing the coding for a His-tag or HQ-tag to give, after any post-translational modifications, the mature polypeptide containing all or part of the His- or HQ-tag. The HQ-tag, having the sequence -RHQHQHQ, may be fully or partly cleaved off the polypeptide during the post-translational modifications resulting in for example the additional amino acids -RHQHQ attached to the N-terminal of the mature polypeptide.

Carbohydrate molecules are often attached to a polypeptide from a fungal source during post-translational modification. In order to aid mass spectrometry analysis, the polypeptide can be incubated with an endoglycosidase to deglycosylate each N-linked position. For every deglycosylated N-linked site, one N-acetyl hexosamine remains on the protein backbone.

Sources of Polypeptides Having GH8 xylanase Activity

A polypeptide having GH8 xylanase activity used in accordance with the present invention may be obtained from any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide GH8 xylanase may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

Polynucleotides

The techniques used to isolate or clone a polynucleotide encoding a polypeptide are known in the art and include isolation from genomic DNA, preparation from cDNA, or a combination thereof.

The cloning of the polynucleotides from such genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Bacillus sp., or another or related organism from the order Bacillales and thus, for example, may be an allelic or species variant of the polypeptide encoding region of the polynucleotide.

Modification of a polynucleotide encoding a polypeptide of the present invention may be necessary for the synthesis of polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. For a general description of nucleotide substitution, see, e.g., Ford et al., 1991, Protein Expression and Purification 2: 95-107.

The Milling Process

The kernels are wet-milled in order to open up the structure and to allow further processing and to separate the kernels into the four main constituents: starch, germ, fiber and protein. Wet milling gives a very good separation of fiber and/or germ and meal (starch granules and protein) and is often applied at locations where there is a parallel production of syrups.

The inventors of the present invention have surprisingly found that the quality of the starch and/or gluten final product may be improved by treating crop kernels in the processes as described herein.

The processes of the invention result in comparison to traditional processes in a higher starch and/or gluten yield and or quality, in that the final starch and gluten product is more pure and/or a higher yield is obtained and/or less process time is used. Another advantage may be that the amount of chemicals, such as SO₂ and NaHSO₃, which need to be used, may be reduced or even fully removed. In terms of processing, it is highly advantageous is the viscosity of the process flow is reduced.

An aspect of the invention relates to a use of a polypeptide having GH8 xylanase activity in a process for treating crop kernels, comprising the steps of:
a) mixing wheat flour and water;
b) adding one or more polypeptide (s) having GH8 xylanase activity;
c) incubating the mixture for a predefined period of time;
d) separating the mixture into two or more fractions including a gluten rich fraction and a starch rich fraction; and
e) recovering the two or more fractions including a gluten rich fraction and a starch rich fraction.

In a preferred embodiment, the polypeptide having GH8 activity is a member of the DPSY clade as defined herein; preferably the polypeptide having GH8 xylanase activity is a member of at least one of the the following clades as defined herein: the SMDY clade, the ALWNW clade, the WFAAAL clade, and the DEAG clade.

Preferably, said GH8 xylanase polypeptide is selected from the group consisting of:
A. a polypeptide having at least 80% sequence identity to the polypeptide of SEQ ID NO: 2, 5, 8, 11, 14, 17, 20 or 23 or the mature polypeptide of SEQ ID NO: 3, 6, 9, 12, 15, 18, 21 or 24;
B. a polypeptide encoded by a polynucleotide that hybridizes under high stringency conditions, or very high stringency conditions with
   (i) the polypeptide coding sequence of SEQ ID NO: 1, 4, 7, 10, 13, 16, 19, or 22;
   (ii) the mature polypeptide coding sequence of SEQ ID NO: 1, 4, 7, 10, 13, 16, 19, or 22;
   (iii) the full-length complementary strand of (i) or (ii);
C. a polypeptide encoded by a polynucleotide having at least 80% sequence identity to the mature polypeptide coding sequence of SEQ ID NO: 1, 4, 7, 10, 13, 16, 19, or 22;
D. a variant of the polypeptide of SEQ ID NO: 2, 5, 8, 11, 14, 17, 20 or 23; or the mature polypeptide of SEQ ID NO: 3, 6, 9, 12, 15, 18, 21 or 24 comprising a substitution, deletion, and/or insertion at one or more (several) positions; and
E. a fragment of a polypeptide of A., B., C., or D having GH8 xylanase activity.

In a preferred embodiment, the polypeptide having GH8 xylanase activity is a polypeptide having at least 85%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 2, 5, 8, 11, 14, 17, 20 or 23; or the mature polypeptide of SEQ ID NO: 3, 6, 9, 12, 15, 18, 21 or 24.

In another preferred embodiment, the polypeptide having GH8 xylanase activity is a polypeptide encoded by a polynucleotide having at least 85%, at least 90%, at least 91%, at least 92%, at least 93%, at least 94%, at least 95%, at least 96%, at least 97%, at least 98%, at least 99% or at least 100% sequence identity to the mature polypeptide coding sequence of SEQ ID NO: 1, 4, 7, 10, 13, 16, 19, or 22.

Alternatively, it is preferred that the polypeptide having GH8 xylanase activity comprises or consists of SEQ ID NO: SEQ ID NO: 2, 5, 8, 11, 14, 17, 20 or 23; or the mature polypeptide of SEQ ID NO: 3, 6, 9, 12, 15, 18, 21 or 24.

In a preferred embodiment, the use further comprises treating the soaked kernels in the presence one or more additional enzyme selected from the group consisting of a beta-xylosidase, cellulase, hemi-celluase, lipase, endoglucanase, acetyl xylan esterase, cellobiohydrolase I, cellobiohydrolase II, and GH61 polypeptide.

Finally, it is preferred that the process provides an improved wheat separation; preferably the process provides a reduced viscosity in wheat flour slurry as determined herein and/or a higher protein recovery as determined herein.

Other Enzymes

The enzyme(s) and polypeptides described below are to be used in an “effective amount” in processes of the present invention. Below should be read in context of the enzyme disclosure in the “Definitions”-section above.

Polypeptides Having Protease Activity

Polypeptides having protease activity, or proteases, are sometimes also designated peptidases, proteinases, peptide hydrolases, or proteolytic enzymes. Proteases may be of the exo-type that hydrolyse peptides starting at either end thereof, or of the endo-type that act internally in polypeptide chains (endopeptidases). Endopeptidases show activity on N- and C-terminally blocked peptide substrates that are relevant for the specificity of the protease in question.

The term “protease” is defined herein as an enzyme that hydrolyses peptide bonds. This definition of protease also applies to the protease-part of the terms “parent protease” and “protease variant,” as used herein. The term “protease” includes any enzyme belonging to the EC 3.4 enzyme group (including each of the eighteen subclasses thereof). The EC number refers to Enzyme Nomenclature 1992 from NC-IUBMB, Academic Press, San Diego, Calif., including supplements 1-5 published in 1994, Eur. J. Biochem. 223: 1-5; 1995, Eur. J. Biochem. 232: 1-6; 1996, Eur. J. Biochem. 237: 1-5; 1997, Eur. J. Biochem. 250: 1-6; and 1999, Eur. J. Biochem. 264: 610-650 respectively. The nomenclature is regularly supplemented and updated; see e.g. the World Wide Web (WWW) at http://www.chem.qmw.ac.uk/iubmb/enzyme/index.html.

The proteases that may be used in a process of the invention could be selected, for example, from:

(a) proteases belonging to the EC 3.4.21. enzyme group; and/or

(b) proteases belonging to the EC 3.4.14. enzyme group; and/or

(c) Serine proteases of the peptidase family S53 that comprises two different types of peptidases: tripeptidyl aminopeptidases (exo-type) and endo-peptidases; as described in 1993, Biochem. J. 290:205-218 and in MEROPS protease database, release, 9.4 (31 Jan. 2011) (www.merops.ac.uk). The database is described in Rawlings, N. D., Barrett, A. J. and Bateman, A., 2010, “MEROPS: the peptidase database”, Nucl. Acids Res. 38: D227-D233.

Cellulolytic Compositions

In an embodiment the cellulolytic composition is derived from a strain of Trichoderma, such as a strain of Trichoderma reesei; a strain of Humicola, such as a strain of Humicola insolens, and/or a strain of Chrysosporium, such as a strain of Chrysosporium lucknowense.

In a preferred embodiment the cellulolytic composition is derived from a strain of Trichoderma reesei.

The cellulolytic composition may comprise one or more of the following polypeptides, including enzymes: GH61 polypeptide having cellulolytic enhancing activity, beta-glucosidase, beta-xylosidase, CBHI and CBHII, endoglucanase, xylanase, or a mixture of two, three, or four thereof.

In an embodiment the cellulolytic composition comprises a GH61 polypeptide having cellulolytic enhancing activity and a beta-glucosidase.

In an embodiment the cellulolytic composition comprises a GH61 polypeptide having cellulolytic enhancing activity and a beta-xylosidase.

In an embodiment, the cellulolytic composition comprises a GH61 polypeptide having cellulolytic enhancing activity and an endoglucanase.

In an embodiment, the cellulolytic composition comprises a GH61 polypeptide having cellulolytic enhancing activity and a xylanase.

In an embodiment, the cellulolytic composition comprises a GH61 polypeptide having cellulolytic enhancing activity, an endoglucanase, and a xylanase.

In an embodiment the cellulolytic composition comprises a GH61 polypeptide having cellulolytic enhancing activity, a beta-glucosidase, and a beta-xylosidase. In an embodiment the cellulolytic composition comprises a GH61 polypeptide having cellulolytic enhancing activity, a beta-glucosidase, and an endoglucanase. In an embodiment the cellulolytic composition comprises a GH61 polypeptide having cellulolytic enhancing activity, a beta-glucosidase, and a xylanase.

In an embodiment the cellulolytic composition comprises a GH61 polypeptide having cellulolytic enhancing activity, a beta-xylosidase, and an endoglucanase. In an embodiment the cellulolytic composition comprises a GH61 polypeptide having cellulolytic enhancing activity, a beta-xylosidase, and a xylanase.

In an embodiment the cellulolytic composition comprises a GH61 polypeptide having cellulolytic enhancing activity, a beta-glucosidase, a beta-xylosidase, and an endoglucanase. In an embodiment the cellulolytic composition comprises a GH61 polypeptide having cellulolytic enhancing activity, a beta-glucosidase, a beta-xylosidase, and a xylanase. In an embodiment the cellulolytic composition comprises a GH61 polypeptide having cellulolytic enhancing activity, a beta-glucosidase, an endoglucanase, and a xylanase.

In an embodiment the cellulolytic composition comprises a GH61 polypeptide having cellulolytic enhancing activity, a beta-xylosidase, an endoglucanase, and a xylanase.

In an embodiment the cellulolytic composition comprises a GH61 polypeptide having cellulolytic enhancing activity, a beta-glucosidase, a beta-xylosidase, an endoglucanase, and a xylanase.

In an embodiment the endoglucanase is an endoglucanase I.

In an embodiment the endoglucanase is an endoglucanase II.

In an embodiment, the cellulolytic composition comprises a GH61 polypeptide having cellulolytic enhancing activity, an endoglucanase I, and a xylanase.

In an embodiment, the cellulolytic composition comprises a GH61 polypeptide having cellulolytic enhancing activity, an endoglucanase II, and a xylanase.

In another embodiment the cellulolytic composition comprises a GH61 polypeptide having cellulolytic enhancing activity, a beta-glucosidase, and a CBHI.

In another embodiment the cellulolytic composition comprises a GH61 polypeptide having cellulolytic enhancing activity, a beta-glucosidase, a CBHI and a CBHII.

The cellulolytic composition may further comprise one or more enzymes selected from the group consisting of an esterase, an expansin, a laccase, a ligninolytic enzyme, a pectinase, a peroxidase, a protease, a swollenin, and a phytase.

GH61 Polypeptide Having Cellulolytic Enhancing Activity

The cellulolytic composition may in one embodiment comprise one or more GH61 polypeptide having cellulolytic enhancing activity.

In one embodiment GH61 polypeptide having cellulolytic enhancing activity, is derived from the genus Thermoascus, such as a strain of Thermoascus aurantiacus, such as the one described in WO 2005/074656 as Sequence Number 2; or a GH61 polypeptide having cellulolytic enhancing activity having at least 80%, such as at least 85%, such such as at least 90%, preferably 95%, such as at least 96%, such as 97%, such as at least 98%, such as at least 99% identity to Sequence Number 2 in WO 2005/074656.

In one embodiment, the GH61 polypeptide having cellulolytic enhancing activity, is derived from a strain derived from Penicillium, such as a strain of Penicillium emersonii, such as the one disclosed in WO 2011/041397, or a GH61 polypeptide having cellulolytic enhancing activity having at least 80%, such as at least 85%, such such as at least 90%, preferably 95%, such as at least 96%, such as 97%, such as at least 98%, such as at least 99% identity to Sequence Number 2 in WO 2011/041397.

In one embodiment the GH61 polypeptide having cellulolytic enhancing activity is derived from the genus Thielavia, such as a strain of Thielavia terrestris, such as the one described in WO 2005/074647 as Sequence Number 7 and Sequence Number 8; or one derived from a strain of Aspergillus, such as a strain of Aspergillus fumigatus, such as the one described in WO 2010/138754 as Sequence Number 2, or a GH61 polypeptide having cellulolytic enhancing activity having at least 80%, such as at least 85%, such such as at least 90%, preferably 95%, such as at least 96%, such as 97%, such as at least 98%, such as at least 99% identity thereto.

Endoglucanase

In one embodiment, the cellulolytic composition comprises an endoglucanase, such as an endoglucanase I or endoglucanase II.

Examples of bacterial endoglucanases that can be used in the processes of the present invention, include, but are not limited to, an Acidothermus cellulolyticus endoglucanase (WO 91/05039; WO 93/15186; U.S. Pat. No. 5,275,944; WO 96/02551; U.S. Pat. No. 5,536,655, WO 00/70031, WO 05/093050); Thermobifida fusca endoglucanase III (WO 05/093050); and Thermobifida fusca endoglucanase V (WO 05/093050).

Examples of fungal endoglucanases that can be used in the present invention, include, but are not limited to, a Trichoderma reesei endoglucanase I (Penttila et al., 1986, Gene 45: 253-263, Trichoderma reesei Cel7B endoglucanase I (GENBANK™ accession no. M15665), Trichoderma reesei endoglucanase II (Saloheimo, et al., 1988, Gene 63:11-22), Trichoderma reesei Cel5A endoglucanase II (GENBANK™ accession no. M19373), Trichoderma reesei endoglucanase III (Okada et al., 1988, Appl. Environ. Microbiol. 64: 555-563, GENBANK™ accession no. AB003694), Trichoderma reesei endoglucanase V (Saloheimo et al., 1994, Molecular Microbiology 13: 219-228, GENBANK™ accession no. Z33381), Aspergillus aculeatus endoglucanase (Ooi et al., 1990, Nucleic Acids Research 18: 5884), Aspergillus kawachii endoglucanase (Sakamoto et al., 1995, Current Genetics 27: 435-439), Erwinia carotovara endoglucanase (Saarilahti et al., 1990, Gene 90: 9-14), Fusarium oxysporum endoglucanase (GENBANK™ accession no. L29381), Humicola grisea var. thermoidea endoglucanase (GENBANK™ accession no. AB003107), Melanocarpus albomyces endoglucanase (GENBANK™ accession no. MAL515703), Neurospora crassa endoglucanase (GENBANK™ accession no. XM_324477), Humicola insolens endoglucanase V, Myceliophthora thermophila CBS 117.65 endoglucanase, basidiomycete CBS 495.95 endoglucanase, basidiomycete CBS 494.95 endoglucanase, Thielavia terrestris NRRL 8126 CEL6B endoglucanase, Thielavia terrestris NRRL 8126 CEL6C endoglucanase, Thielavia terrestris NRRL 8126 CEL7C endoglucanase, Thielavia terrestris NRRL 8126 CEL7E endoglucanase, Thielavia terrestris NRRL 8126 CEL7F endoglucanase, Cladorrhinum foecundissimum ATCC 62373 CEL7A endoglucanase, and Trichoderma reesei strain No. VTT-D-80133 endoglucanase (GENBANK™ accession no. M15665).

In one embodiment, the endoglucanase is an endoglucanase II, such as one derived from Trichoderma, such as a strain of Trichoderma reesei, such as the one described in WO 2011/057140 as Sequence Number 22; or an endoglucanase having at least 80%, such as at least 85%, such such as at least 90%, preferably 95%, such as at least 96%, such as 97%, such as at least 98%, such as at least 99% identity to Sequence Number 22 in WO 2011/057140. In one aspect, the protease differs by up to 10 amino acids, e.g., 1, 2, 3, 4, 5, 6, 7, 8, 9, or 10, from the mature polypeptide of Sequence Number 22 in WO 2011/057140. In another embodiment, the present invention relates to variants of the mature polypeptide of Sequence Number 22 in WO 2011/057140 comprising a substitution, deletion, and/or insertion at one or more (e.g., several) positions. In an embodiment, the number of amino acid substitutions, deletions and/or insertions introduced into the mature polypeptide of Sequence Number 22 in WO 2011/057140 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function.

Beta-Xylosidase

Examples of beta-xylosidases useful in the processes of the present invention include, but are not limited to, beta-xylosidases from Neurospora crassa (SwissProt accession number Q7SOW4), Trichoderma reesei (UniProtKB/TrEMBL accession number Q92458), and Talaromyces emersonii (SwissProt accession number Q8X212).

In one embodiment the beta-xylosidase is derived from the genus Aspergillus, such as a strain of Aspergillus fumigatus, such as the one described in WO 2011/057140 as Sequence Number 206; or a beta-xylosidase having at least 80%, such as at least 85%, such such as at least 90%, preferably 95%, such as at least 96%, such as 97%, such as at least 98%, such as at least 99% identity to Sequence Number 206 in WO 2011/057140. In one aspect, the protease differs by up to 10 amino acids, e.g., 1, 2, 3, 4, 5, 6, 7, 8, 9, or 10, from the mature polypeptide of SEQ ID NO: 206 described in WO 2011/057140. In another embodiment, the present invention relates to variants of the mature polypeptide of SEQ ID NO: 206 described in WO 2011/057140 comprising a substitution, deletion, and/or insertion at one or more (e.g., several) positions. In an embodiment, the number of amino acid substitutions, deletions and/or insertions introduced into the mature polypeptide of SEQ ID NO: 206 described in WO 2011/057140 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function.

In one embodiment the beta-xylosidase is derived from a strain of the genus Aspergillus, such as a strain of Aspergillus fumigatus, such as the one disclosed in U.S. provisional No. 61/526,833 or PCT/US12/052163 (Examples 16 and 17), or derived from a strain of Trichoderma, such as a strain of Trichoderma reesei, such as the mature polypeptide of Sequence Number 58 in WO 2011/057140 or a beta-xylosidase having at least 80%, such as at least 85%, such such as at least 90%, preferably 95%, such as at least 96%, such as 97%, such as at least 98%, such as at least 99% identity thereto.

Beta-Glucosidase

The cellulolytic composition may in one embodiment comprise one or more beta-glucosidase. The beta-glucosidase may in one embodiment be one derived from a strain of the genus Aspergillus, such as Aspergillus oryzae, such as the one disclosed in WO 2002/095014 or the fusion protein having beta-glucosidase activity disclosed in WO 2008/057637, or Aspergillus fumigatus, such as such as one disclosed in WO 2005/047499 or an Aspergillus fumigatus beta-glucosidase variant, such as one disclosed in PCT application PCT/US11/054185 (or U.S. provisional application No. 61/388,997), such as one with the following substitutions: F100D, S283G, N456E, F512Y.

In one embodiment the beta-glucosidase is derived from the genus Aspergillus, such as a strain of Aspergillus fumigatus, such as the one described in WO 2005/047499, or a beta-glucosidase having at least 80%, such as at least 85%, such such as at least 90%, preferably 95%, such as at least 96%, such as 97%, such as at least 98%, such as at least 99% identity thereto.

In one embodiment the beta-glucosidase is derived from the genus Aspergillus, such as a strain of Aspergillus fumigatus, such as the one described in WO 2012/044915, or a beta-xylosidase having at least 80%, such as at least 85%, such such as at least 90%, preferably 95%, such as at least 96%, such as 97%, such as at least 98%, such as at least 99% identity thereto.

Cellobiohydrolase I

The cellulolytic composition may in one embodiment may comprise one or more CBH I (cellobiohydrolase I). In one embodiment the cellulolytic composition comprises a cellobiohydrolase I (CBHI), such as one derived from a strain of the genus Aspergillus, such as a strain of Aspergillus fumigatus, such as the Cel7A CBHI disclosed in Sequence Number 2 in WO 2011/057140, or a strain of the genus Trichoderma, such as a strain of Trichoderma reesei.

In one embodiment the cellobiohydrolyase I is derived from the genus Aspergillus, such as a strain of Aspergillus fumigatus, such as the one described in WO 2011/057140, or a CBHI having at least 80%, such as at least 85%, such such as at least 90%, preferably 95%, such as at least 96%, such as 97%, such as at least 98%, such as at least 99% identity thereto.

Cellobiohydrolase 11

The cellulolytic composition may in one embodiment comprise one or more CBH II (cellobiohydrolase II). In one embodiment the cellobiohydrolase II (CBHII), such as one derived from a strain of the genus Aspergillus, such as a strain of Aspergillus fumigatus, or a strain of the genus Trichoderma, such as Trichoderma reesei, or a strain of the genus Thielavia, such as a strain of Thielavia terrestris, such as cellobiohydrolase II CEL6A from Thielavia terrestris.

In one embodiment the cellobiohydrolyase II is derived from the genus Aspergillus, such as a strain of Aspergillus fumigatus, such as the one described in WO 2011/057140, or a CBHII having at least 80%, such as at least 85%, such such as at least 90%, preferably 95%, such as at least 96%, such as 97%, such as at least 98%, such as at least 99% identity thereto.

Exemplary Cell ulolytic Compositions

As mentioned above the cellulolytic composition may comprise a number of different polypeptides, such as enzymes.

In an embodiment, the cellulolytic composition comprises a Trichoderma reesei cellulase preparation containing Aspergillus oryzae beta-glucosidase fusion protein (e.g. SEQ ID NO: 74 or 76 in WO 2008/057637) and Thermoascus aurantiacus GH61A polypeptide (e.g., SEQ ID NO: 2 in WO 2005/074656).

In an embodiment, the cellulolytic composition comprises a blend of an Aspergillus aculeatus GH10 xylanase (e.g., SEQ ID NO: 5 (Xyl II) in WO 94/021785) and a Trichoderma reesei cellulase preparation containing Aspergillus fumigatus beta-glucosidase (e.g., SEQ ID NO: 2 in WO 2005/047499) and Thermoascus aurantiacus GH61A polypeptide (e.g., SEQ ID NO: 2 in WO 2005/074656).

In an embodiment, the cellulolytic composition comprises a blend of an Aspergillus fumigatus GH10 xylanase (e.g., SEQ ID NO: 6 (Xyl III) in WO 2006/078256) and Aspergillus fumigatus beta-xylosidase (e.g., SEQ ID NO: 206 in WO 2011/057140) with a Trichoderma reesei cellulase preparation containing Aspergillus fumigatus cellobiohydrolase I (e.g., SEQ ID NO: 6 in WO 2011/057140), Aspergillus fumigatus cellobiohydrolase II (e.g., SEQ ID NO: 18 in WO 2011/057140), Aspergillus fumigatus beta-glucosidase variant (e.g., one having F100D, S283G, N456E, F512Y substitutions disclosed in WO 2012/044915), and Penicillium sp. (emersonii) GH61 polypeptide (e.g., SEQ ID NO: 2 in WO 2011/041397).

In an embodiment the cellulolytic composition comprises a Trichoderma reesei cellulolytic enzyme composition, further comprising Thermoascus aurantiacus GH61A polypeptide having cellulolytic enhancing activity (e.g., SEQ ID NO: 2 in WO 2005/074656) and Aspergillus oryzae beta-glucosidase fusion protein (e.g., SEQ ID NO: 74 or 76 in WO 2008/057637).

In another embodiment the cellulolytic composition comprises a Trichoderma reesei cellulolytic enzyme composition, further comprising Thermoascus aurantiacus GH61A polypeptide having cellulolytic enhancing activity (e.g., SEQ ID NO: 2 in WO 2005/074656) and Aspergillus fumigatus beta-glucosidase (e.g., SEQ ID NO: 2 in WO 2005/047499).

In another embodiment the cellulolytic composition comprises a Trichoderma reesei cellulolytic enzyme composition, further comprising Penicillium emersonii GH61A polypeptide having cellulolytic enhancing activity disclosed as, e.g., SEQ ID NO: 2 in WO 2011/041397, Aspergillus fumigatus beta-glucosidase (e.g., SEQ ID NO: 2 in WO 2005/047499) or a variant thereof with the following substitutions: F100D, S283G, N456E, F512Y.

The enzyme composition of the present invention may be in any form suitable for use, such as, for example, a crude fermentation broth with or without cells removed, a cell lysate with or without cellular debris, a semi-purified or purified enzyme composition, or a host cell, e.g., Trichoderma host cell, as a source of the enzymes.

The enzyme composition may be a dry powder or granulate, a non-dusting granulate, a liquid, a stabilized liquid, or a stabilized protected enzyme. Liquid enzyme compositions may, for instance, be stabilized by adding stabilizers such as a sugar, a sugar alcohol or another polyol, and/or lactic acid or another organic acid according to established processes.

Enzymatic Amount

In particular embodiments, the GH8 xylanase is present in the enzyme composition in a range of about 5% w/w to about 65% w/w of the total amount of enzyme protein. In other embodiments, the protease is present in about 5% w/w to about 60% w/w, about 5% w/w to about 50% w/w, about 5% w/w to about 40% w/w, about 5% w/w to about 30% w/w, about 10% w/w to about 30% w/w, or about 10% w/w to about 20% w/w.

Enzymes may be added in an effective amount, which can be adjusted according to the practitioner and particular process needs. In general, enzyme may be present in an amount of 0.0001-2.5 mg total enzyme protein per g dry solids (DS) kernels, preferably 0.001-1 mg enzyme protein per g DS kernels, preferably 0.0025-0.5 mg enzyme protein per g DS kernels, preferably 0.025-0.25 mg enzyme protein per g DS kernels, preferably 0.05-0.125 mg enzyme protein per g DS kernels. In particular embodiments, the enzyme may be present in an amount of, e.g. 2.5 μg, 12.5 μg, 25 μg, 50 μg, 75 μg, 100 μg, 125 μg, 150 μg, 175 μg, 200 μg, 250 μg, 500 μg enzyme protein per g DS kernels.

Other Enzyme Activities

According to the invention an effective amount of one or more of the following activities may also be present or added during treatment of the kernels: pentosanase, pectinase, arabinanase, arabinofurasidase, xyloglucanase, phytase activity.

It is believed that after the division of the kernels into finer particles the enzyme(s) can act more directly and thus more efficiently on cell wall and protein matrix of the kernels. Thereby the starch is washed out more easily in the subsequent steps.

### EXAMPLES

### Example 1

### Strains and DNA

Genes encoding a number of GH8 xylanases or GH8 xylanase domains were isolated from bacterial strains and environmental bacterial communities isolated from soil samples collected in Denmark and in the United States (see table 1).

Chromosomal DNA from the different strains and bacterial communities was subjected to full genome sequencing. The genome sequences were analyzed for glycosyl hydrolase domains (according to the CAZY definition). A number of glycosyl hydrolase family 8 (GH8) xylanase or xylanase-domain coding sequences were identified. Some of these were part or larger multidomain enzymes with, for example one or more C-terminal carbohydrate-binding domain (CBM). For the purposes of the instant invention only the mature GH8 xylanase domains were expressed, with the exception of SEQ ID NO:6 which was expressed and tested with its native C-terminal CBM.

One wildtype GH8 xylanase-encoding gene from Bacillus sp. KK-1 also disclosed in WO 2011/070101 (Novozymes) was modified to encode a variant GH xylanase having a single leucine insertion, N→NL, in position 82 of the full-length polypeptide (as shown in SEQ ID NO:2) which is position 55 in the mature polypeptide (as shown in SEQ ID NO:3).

TABLE 1
List of GH8 xylanases and their origin.

| | | country of |
|---|---|---|
| SEQ ID NO | Donor | origin |
| SEQ ID NO: 1-3 | Insertion variant (N82NL; N55NL) | Denmark |
| | Bacillus sp. KK-1 GH8 xylanase | |
| SEQ ID NO: 4-6 | Dyella sp-62206 | Denmark |
| SEQ ID NO: 7-9 | Bacillus licheniformis | Denmark |
| SEQ ID NO: 10-12 | Marinimicrobium sp-62868 | United States |
| SEQ ID NO: 13-15 | Marinimicrobium sp-62335 | Denmark |
| SEQ ID NO: 16-18 | Metagenome from environmental | Denmark |
| | sample C | |
| SEQ ID NO: 19-21 | Metagenome from environmental | Denmark |
| | sample C | |
| SEQ ID NO: 22-24 | Metagenome from environmental | Denmark |
| | sample K | |

### Example 2

### Expression of GH8 Xylanases

A linear integration vector-system was used for the expression cloning of the different GH8 xylanases shown in Table 1. The linear integration construct was a PCR fusion product containing the respective xylanase domain-encoding polynucleotide operably linked with a strong promoter and a chloramphenicol resistance selectable marker flanked between two Bacillus subtilis homologous chromosomal regions. The fusion was made by SOE PCR (Horton, R. M., Hunt, H. D., Ho, S. N., Pullen, J. K. and Pease, L. R. (1989) Engineering hybrid genes without the use of restriction enzymes, gene splicing by overlap extension Gene 77: 61-68). Suitable strong promoters are described in WO 1999/43835. The chloramphenicol acetyl-transferase resistance marker gene was described in e.g. Diderichsen, B.; Poulsen, G. B.; Joergensen, S. T. 1993, Plasmid, “A useful cloning vector for Bacillus subtilis” 30:312. The final gene constructs were integrated on the Bacillus subtilis chromosome by homologous recombination into the pectate lyase locus.

The genes encoding the GH8 xylanases or domains were amplified from chromosomal DNA using gene specific primers containing overhangs to the two flanking fragments. The GH8 xylanases were expressed with a Bacillus clausii secretion signal (with the following amino acid sequence: MKKPLGKIVASTALLISVAFSSSIASA; SEQ ID NO:25) replacing the native secretion signals and with a 6x histidine tag fused directly to the C-terminal of the protein for later protein chromatography column-purification.

The two linear vector fragments and the gene fragments were subjected to a Splicing by Overlap Extension (SOE) PCR reaction to assemble the 3 fragments into one linear vector construct for each gene. An aliquot of the PCR product was then transformed into a Bacillus subtilis host cell. Transformants were selected on LB plates supplemented with 6 μg of chloramphenicol per ml. A recombinant Bacillus subtilis clone from each construct containing the integrated expression construct was cultivated in 3L flasks containing 500 ml yeast extract-based medium at 30° C. for 4 days with shaking at 250 rpm. Each of the culture broths were centrifuged at 20,000×g for 20 minutes and the supernatants were carefully decanted from the pelleted material. Each supernatant was filtered using a filtration unit equipped with a 0.2 μm filter (Nalgene) to remove any cellular debris. The enzymes were purified from the filtered supernatant as described in Example 3.

### Example 3

### Purification of the GH8 Xylanases

The GH8 xylanases were purified in the following way: The pH of the supernatant was adjusted to pH 8 with 3 M Tris, left for 1 hour, and then filtered using a filtration unit equipped with a 0.2 μm filter (Nalgene). The filtered supernatant was applied to a 5 ml HisTrap™ Excel column (GE Healthcare Life Sciences) pre-equilibrated with 5 column volumes (CV) of 50 mM Tris/HCl pH 8. Unbound protein was eluted by washing the column with 8 CV of 50 mM Tris/HCl pH 8.

The xylanases were eluted with 50 mM HEPES pH 7-10 mM imidazole and elution was monitored by absorbance at 280 nm. The eluted xylanases were desalted on a HiPrep™ 26/10 desalting column (GE Healthcare Life Sciences) pre-equilibrated with 3 CV of 50 mM HEPES pH 7-100 mM NaCl. The xylanases were eluted from the column using the same buffer at a flow rate of 10 ml/minute. Relevant fractions were selected and pooled based on the chromatogram and SDS-PAGE analysis using 4-12% Bis-Tris gels (Invitrogen) and 2-(N-morpholino)ethanesulfonic acid (MES) SDS-PAGE running buffer (Invitrogen). The gel was stained with InstantBlue (Novexin) and destained using miliQ water. The concentrations of the purified enzymes were determined by absorbance at 280 nm.

### Example 4

### Construction of GH8 Xylanase Phylogenetic Trees

The GH8 family, which includes the xylanases of the invention, may be sub-divided into clusters or clades. A phylogenetic tree was constructed, of polypeptide sequences containing a GH8 domain, as defined in CAZY (Carbohydrate Active Enzymes database, http://www.cazy.org/, Henrissat et al, 2014, Nucleic Acids Res 42:D490—D495). The phylogenetic tree was constructed from a multiple alignment of mature polypeptide sequences containing at least one GH8 domain. The sequences were aligned using the MUSCLE algorithm version 3.8.31 (Edgar, 2004. Nucleic Acids Research 32(5): 1792-1797), and the tree were constructed using FastTree version 2.1.8 (Price et al., 2010, PloS one 5(3)) and visualized using iTOL (Letunic & Bork, 2007. Bioinformatics 23(1): 127-128).

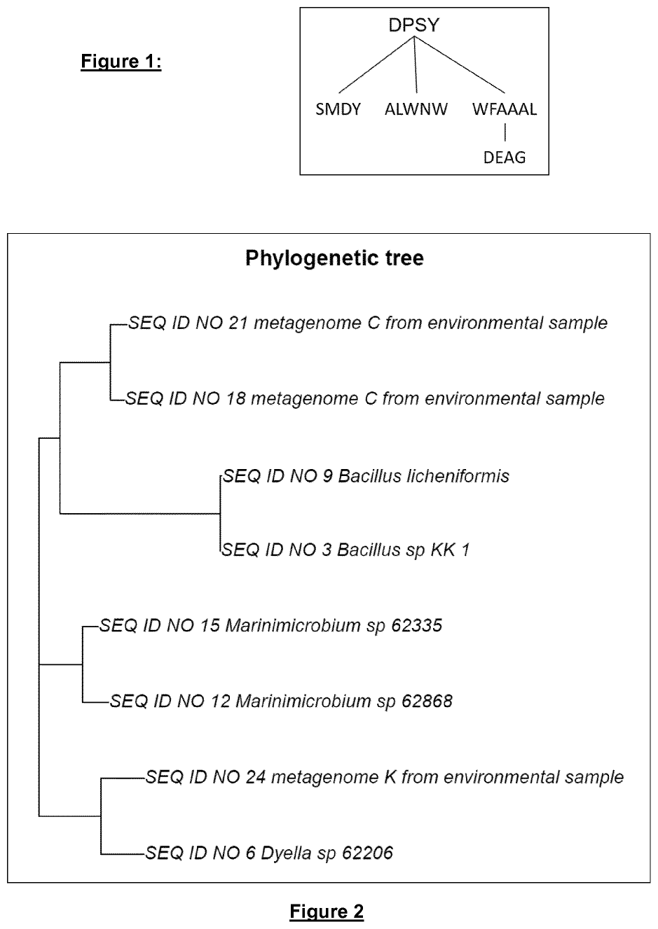

The polypeptides in GH8 can be separated into multiple distinct sub-clusters, or clades, where we denoted the clades listed below. Distinct motifs for each clade are described in details below and illustrated in FIG. 1.
(a) DPSY clade
(b) SMDY clade
(c) ALWNW clade
(d) WFAAAL clade
(e) DEAG clade
The DPSY Clade

GH8 xylanases comprise several well-conserved motifs, one example is the motif “[TS]D[PA]SY” or “(Thr/Ser) Asp (Pro/Ala) Ser Tyr” (SEQ ID NO: 26) situated in positions 204-207 of the xylanase amino acid sequence shown in SEQ ID NO:2 and 3, in positions 203-206 of SEQ ID NO:8 and 9, in positions 342-345 of SEQ ID NO:11 and 12, in positions 342-345 of SEQ ID NO:14 and 15, in positions 194-197 of SEQ ID NO:17 and 18, and in positions 201-204 of SEQ ID NO:20 and 21. We denote one sub-cluster or clade of GH8 xylanases comprising the motif ““[TS]D[PA]SY”” or “(Thr/Ser) Asp (Pro/Ala) Ser Tyr” (SEQ ID NO:26) the DPSY clade.

The SMDY clade

A phylogenetic tree was constructed of polypeptide sequences containing the GH8 polypeptides from the DPSY clade, as defined above. The phylogenetic tree was constructed from a multiple alignment of mature polypeptide sequences containing at least one GH8 domain. The sequences were aligned using the MUSCLE algorithm version 3.8.31 (Edgar, 2004. Nucleic Acids Research 32(5): 1792-1797), and the tree was constructed using FastTree version 2.1.8 (Price et al., 2010, PloS one 5(3)) and visualized using iTOL (Letunic & Bork, 2007. Bioinformatics 23(1): 127-128). The polypeptides of the DPSY clade can be separated into distinct sub-clusters, and one of the sub-clusters we denote “SMDY”. A characteristic motif for this subgroup is the motif “MN[FYVILM][GS]MDY” or “Met Asn (Phe/Tyr/Val/Ile/Leu/Met) (Gly/Ser) Met Asp Tyr” (SEQ ID NO:27). This motif is found in amino acid positions 277 to 283 of the xylanase amino acid sequence shown in SEQ ID NO:20 and 21.

The ALWNW Clade

A phylogenetic tree was constructed, of polypeptide sequences containing the GH8 polypeptides from the DPSY clade, as defined above. The phylogenetic tree was constructed from a multiple alignment of mature polypeptide sequences containing at least one GH8 domain. The sequences were aligned using the MUSCLE algorithm version 3.8.31 (Edgar, 2004. Nucleic Acids Research 32(5): 1792-1797), and the tree was constructed using FastTree version 2.1.8 (Price et al., 2010, PloS one 5(3)) and visualized using iTOL (Letunic & Bork, 2007. Bioinformatics 23(1): 127-128). The polypeptides of the DPSY clade can be separated into distinct sub-clusters, and one of the sub-clusters we denote “ALWNW”. A characteristic motif for this subgroup is the motif “A[IL]WNW” or “Ala (Ile/Leu) Trp Asn Trp” (SEQ ID NO:28) corresponding to positions 101 to 105 of the xylanase amino acid sequence shown in SEQ ID NO:5 and 6, and to positions 101 to 105 in SEQ ID NO:23 and 24.

The WFAAAL clade

A phylogenetic tree was constructed, of polypeptide sequences containing the GH8 polypeptides from the DPSY clade, as defined above. The phylogenetic tree was constructed from a multiple alignment of mature polypeptide sequences containing at least one GH8 domain. The sequences were aligned using the MUSCLE algorithm version 3.8.31 (Edgar, 2004. Nucleic Acids Research 32(5): 1792-1797), and the tree was constructed using FastTree version 2.1.8 (Price et al., 2010, PloS one 5(3)) and visualized using iTOL (Letunic & Bork, 2007. Bioinformatics 23(1): 127-128). The polypeptides of the DPSY clade can be separated into distinct sub-clusters, and one of the sub-clusters we denote “WFAAAL”. A characteristic motif for this subgroup is the motif “W[IF]AAAL” or “Trp (Ile/Phe) Ala Ala Ala Leu” (SEQ ID NO:29) corresponding to positions 134 to 139 of the xylanase amino acid sequence shown in SEQ ID NO:2 and 3, and to positions. 133 to 138 in SEQ ID NO:8 and 9

The DEAG Clade

A phylogenetic tree was constructed, of polypeptide sequences containing the GH8 polypeptides from the WFAAAL clade, as defined above. The phylogenetic tree was constructed from a multiple alignment of mature polypeptide sequences containing at least one GH8 domain. The sequences were aligned using the MUSCLE algorithm version 3.8.31 (Edgar, 2004. Nucleic Acids Research 32(5): 1792-1797), and the tree was constructed using FastTree version 2.1.8 (Price et al., 2010, PloS one 5(3)) and visualized using iTOL (Letunic & Bork, 2007. Bioinformatics 23(1): 127-128). The polypeptides of the DPSY clade can be separated into distinct sub-clusters, and one of the sub-clusters we denote “DEAG”. A characteristic motif for this subgroup is the motif “DEAG” or “Asp Glu Ala Gly” (SEQ ID NO:30) corresponding to the amino acids in positions 264 to 267 of the xylanase amino acid sequence shown in SEQ ID NO:2 and 3.

Another motif is “AANAGGA” or “Ala Ala Asn Ala Gly Gly Ala” (SEQ ID NO:31), corresponding to the amino acids in positions 354 to 360 of the xylanase amino acid sequence shown in SEQ ID NO:2 and 3, and in positions 352 to 358 of SEQ ID NO:8 and 9.

A phylogenetic tree of the polypeptides of the invention is shown in FIG. 2.

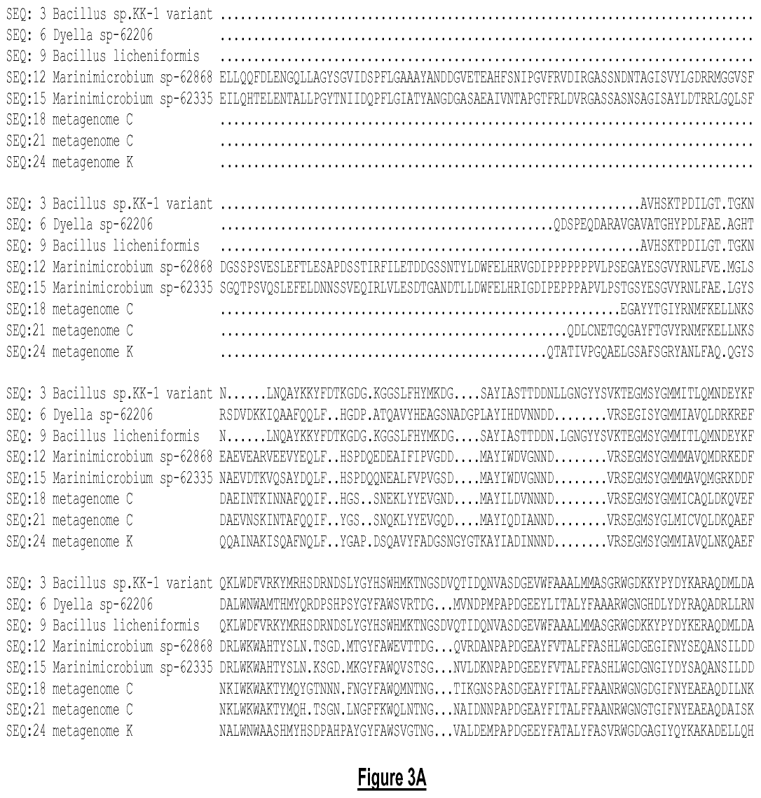

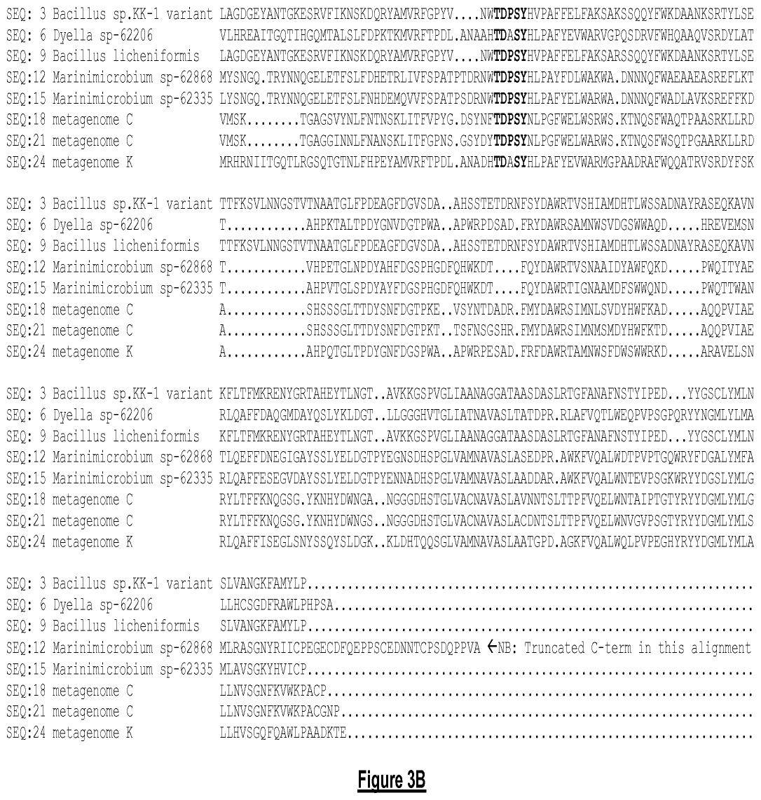

An alignment of the GH8 xylanase amino acid sequences herein is shown in FIG. 3.

### Example 5

### Determining Xylanase-Catalyzed Viscosity Change in Wheat Flour Slurry

Viscosity reduction catalyzed by the GH8 xylanases of the invention was determined by using the Viscosity-Pressure (ViPr) assay disclosed in WO 2011/107472. Those xylanases that hydrolyze or alter components that contribute directly or indirectly to the viscosity of a wheat flour slurry are identified by measuring viscosity changes during or after incubation.

Substrate Preparation

80 g wheat flour was sieved through 4 consecutive sieves of: 800 μm, 600 μm, 400 μm and 300 μm. A slurry of Wheat Flour was prepared by mixing sieved flour under continuous & rigid stirring into a solution of MilliQ water (0.76 mM CaCl2) to reach a dry-solids concentration (DS) of 30% with the pH adapted to 6 by addition of 1.6 M HCl.

5 ml of the wheat slurry was added to each well of a 24 well plate (10 ml volume per well, round bottom). The dispersion was stirred at room temperature using cross bar magnets (9 mm). 20 μl aliquots of diluted Enzyme solution prepared with MilliQ water was added to each well reaching a dose of 4,4 μg or 1.95 μg Enzyme Protein per g dry substance (DS). Suitable pipette tips for the ViPr measurements were produced by removing 12 cm of the lower part of a 1 ml tip, sliding a silicone tube (outer diameter 8 mm; 2 mm length) on to the remaining tip and pushing a wide-bore tip (Sartorius 791020) over the silicon tube.

The viscosity was measured on the Hamilton STAR® liquidhandler every second minute with the following parameters; 20 μl enzyme or control was added after the 4 minutes right after second timepoint viscosity measurement.

Hamilton STAR® liquidhandler settings:

Time points: 30; Interval: 120 sec; Repetition: 3, Aspiration height: 16 mm, Dispense height: 16 mm; Liquidclass—flow rate Aspirate and Dispense: 500 μl/s

First 200 μl air was aspirated followed by immersing the adapted ViPr pipette tip into the slurry and aspirating 800 μl. The 800 μl slurry was dispensed back to the remaining slurry with the tip above the liquid and finally the 200 μl air was used to blow out remaining liquid.

Pressure readings were extracted from the Hamilton TADM data file and timepoint 1000 ms on the pressure curve during dispense was used for further data analysis.

The viscosity change was expressed by the percental change of the pressure value in the enzyme treated samples compared to control samples.

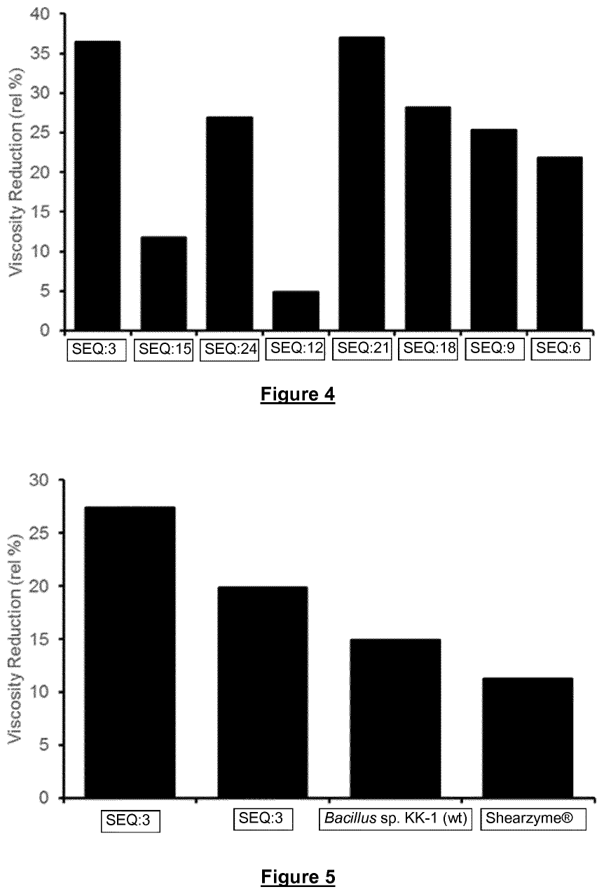

FIG. 4 shows that all eight GH8 xylanases, all members of the DPSY clade as defined above, are effective at reducing the viscosity of the wheat slurry.

FIG. 5 shows the wheat slurry viscosity reduction by two of the GH8 xylanase clade “DPSY” members; one is the parent wildtype GH8 xylanase from Bacillus sp. KK-1 and the other, which is even better at reducing the viscosity, is the single leucine insertion variant of SEQ ID NO:3. The latter was tested with enzyme from two separate production batches. Both the parent and the variant GH8 xylanases are better at reducing the viscosity than a commercially available GH10 xylanase (Shearzyme®, Novozymes).

### Example 6

### Wheat Protein Recovery

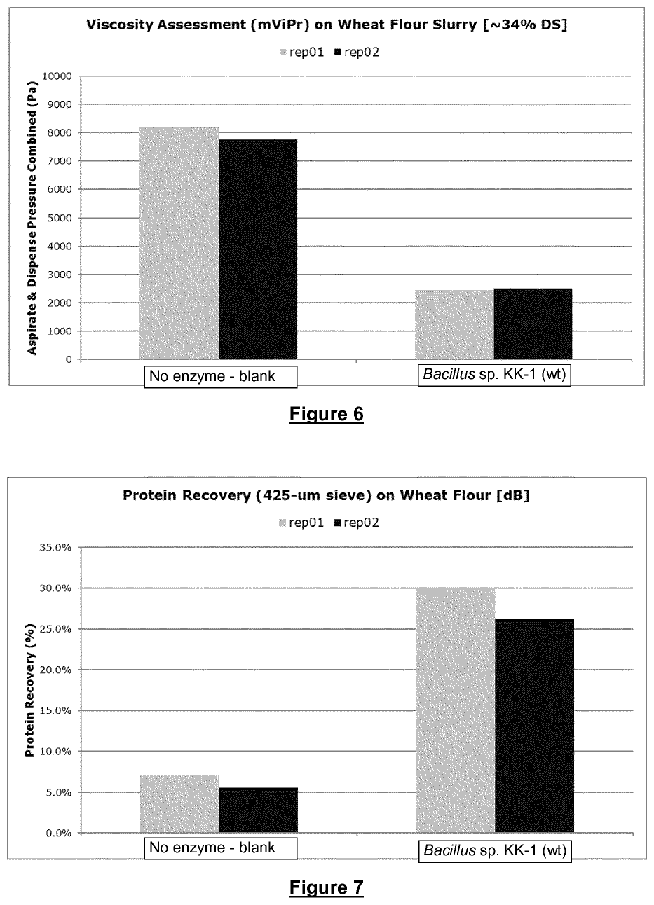

Approximately 250 g of wheat flour and 150 mL of heated tap water (containing the Bacillus sp. KK-1 wildtype GH8 xylanase enzyme if applicable) were transferred, respectively, into an appropriately sized mixing bowl and mixed for 4 minutes with a Kitchen Aid Ultra Power (300 watts max) stand-mixer equipped with a dough hook and set to a speed of 4. Afterwards, the formed dough was allowed to rest for 8 minutes, then 250 mL of heated tap water was added to the mixing bowl. The contents were mixed for an additional 25 minutes with a flat beater at a speed setting of stir. Approximately 5 mL of the resultant slurry were removed for viscosity assessment. The results are shown in FIG. 6, where it is clear that the Bacillus sp. KK-1 wildtype GH8 xylanase reduced the viscosity of the wheat slurry suprisingly about 4-fold. Then 1000 mL of heated tap water was added to the mixing bowl. The contents were stirred again for 35 minutes, then poured over a 425-um sieve. The sieve was vibrated to enable separation. Approximately 1000 mL of heated tap water was added to the mixing bowl for a final rinse, then poured over said sieve and vibrated as before. The material remaining on top of the sieve was recovered and then analyzed for protein content using a total nitrogen analyzer (LECO corporation model FP628). The results are shown in FIG. 7, where it is clear that the Bacillus sp. KK-1 wildtype GH8 xylanase surprisingly improved the protein recovery from about 5% to 25-30%, i.e., close to a 6-fold improvement.

1. A process for separating wheat flour into two or more fractions including a gluten fraction and a starch fraction, comprising the steps of:
a) mixing wheat flour and water;
b) adding one or more polypeptide(s) having GH8 xylanase activity;
c) incubating the mixture for a predefined period of time;
d) separating the mixture into two or more fractions including a gluten rich fraction and a starch rich fraction; and
e) recovering the two or more fractions including a gluten rich fraction and a starch rich fraction.

2. The process of claim 1, wherein the polypeptide(s) having GH8 xylanase activity is (are) a member of the DPSY clade as defined herein; preferably the polypeptide having GH8 xylanase activity is a member of at least one of the following clades as defined herein: the SMDY clade, the ALWNW clade, the WFAAAL clade, and the DEAG clade.

3. The process of claim 1 or 2, wherein the polypeptide having GH8 xylanase activity is selected from the group consisting of:
A. a polypeptide having at least 80% sequence identity to the polypeptide of SEQ ID NO: 2, 5, 8, 11, 14, 17, 20 or 23 or the mature polypeptide of SEQ ID NO: 3, 6, 9, 12, 15, 18, 21 or 24;
B. a polypeptide encoded by a polynucleotide that hybridizes under high stringency conditions, or very high stringency conditions with
(i) the polypeptide coding sequence of SEQ ID NO: 1, 4, 7, 10, 13, 16, 19, or 22;
(ii) the mature polypeptide coding sequence of SEQ ID NO: 1, 4, 7, 10, 13, 16, 19, or 22;
(iii) the full-length complementary strand of (i) or (ii);
C. a polypeptide encoded by a polynucleotide having at least 80% sequence identity to the mature polypeptide coding sequence of SEQ ID NO: 1, 4, 7, 10, 13, 16, 19, or 22;
D. a variant of the polypeptide of SEQ ID NO: 2, 5, 8, 11, 14, 17, 20 or 23; or the mature polypeptide of SEQ ID NO: 3, 6, 9, 12, 15, 18, 21 or 24 comprising a substitution, deletion, and/or insertion at one or more (several) positions; and
E. a fragment of a polypeptide of A., B., C., or D having GH8 xylanase activity.

4. The process of claim 3, wherein the polypeptide having GH8 activity is a polypeptide having at least 85% sequence identity to the polypeptide of SEQ ID NO: 2, 5, 8, 11, 14, 17, 20 or 23; or the mature polypeptide of SEQ ID NO: 3, 6, 9, 12, 15, 18, 21 or 24.

5. The process of claim 3, wherein the polypeptide having GH8 xylanase activity is a polypeptide encoded by a polynucleotide having at least 85% sequence identity to the mature polypeptide coding sequence of SEQ ID NO: 1, 4, 7, 10, 13, 16, 19, or 22.

6. The process of claim 3, wherein the polypeptide having GH8 xylanase activity comprises or consists of SEQ ID NO: 2, 5, 8, 11, 14, 17, 20 or 23;
or the mature polypeptide of SEQ ID NO: 3, 6, 9, 12, 15, 18, 21 or 24.

7. The process of claim 1, further comprising treating the soaked kernels in the presence of one or more enzyme selected from the group consisting of a beta-xylosidase, cellulase, hemi-celluase, lipase, endoglucanase, acetylan esterase, cellobiohydrolase I, cellobiohydrolase II, and GH61 polypeptide.

8. The process of claim 1, wherein said polypeptide is present in an amount of preferably 0.0005 to 1.5 mg enzyme protein per g DS kernels.

9. The process of claim 1, where in step a) the water and wheat flour are mixed in a ratio of 0.1-3 Liter of water per kg wheat flour.

10. The process of claim 1, wherein the incubation in step c) is performed for 5 minutes to 8 Hours.

11. The process of claim 1, wherein step d) is performed in a three-phase separator and provides a gluten rich fraction, a starch rich fraction and a pentosane/fiber rich fraction.

12. The process of claim 1, wherein an improved wheat separation is provided.

13-20. (canceled)
